(19) United States
(12) Patent Application Publication
(10) Pub. No.: US 2021/0295448 A1
(43) Pub. Date: Sep. 23, 2021

(54) SYSTEMS AND METHODS FOR DETERMINING ELIGIBILTY AND CONTRIBUTIONS FOR SERVICES AND/OR PRODUCTS ACROSS MULTIPLE PLATFORMS

(71) Applicant: McClain Investments LLC, Boise, ID (US)

(72) Inventors: Nathan Alan McClain, Gilbert, AZ (US); Brittany Ann Kellams-McClain, Gilbert, AZ (US); Jonathan Jeremy Cardella, Boise, ID (US); Roman Yevgeniyvich Kolyvanov, Boise, ID (US); Andrew Ray Palmer, Boise, ID (US); Vladimir Malkhazovich Dzhidzhiyeshvili, Boise, ID (US); Long Hoang Nguyen, Ho Chi Minh City (VN)

(21) Appl. No.: 17/205,984

(22) Filed: Mar. 18, 2021

Related U.S. Application Data
(60) Provisional application No. 62/991,264, filed on Mar. 18, 2020.

(51) Int. Cl. G06Q 40/08; G16H 10/60; G06N 5/02

(52) U.S. Cl. CPC G06Q 40/08; G06N 5/025; G16H 10/60

(57) ABSTRACT

Systems and methods for identifying eligibility and contribution level for services and/or products across multiple insurance platforms for provisioning benefit (and non-benefit) services and products to a patient. Benefits plan details for a plurality of benefit plans, and from a plurality of insurers or benefits management entities can be acquired and mapped to an inventory of products and/or services by one or more service providers. Business logic derived from the benefits plan details and the business logic can be used to read a personal benefits plan of an individual and determine personalized pricing for one or more of the inventory products. The benefit plan details and provider inventory items are correlated for a particular patient to render in near real-time available benefits, corresponding services, and patient-borne costs based directly on the benefits plan under which the particular patient may be covered.

### RELATED APPLICATIONS

The present application claims the benefit of priority under 35 U.S.C. Section 119(e) of U.S. Provisional Patent Application No. 62/991,264 entitled SYSTEMS AND METHODS FOR IDENTIFYING SERVICE AND/OR PRODUCT ELIGIBILITY AND PRICING ACROSS MULTIPLE INSURANCE PLATFORMS, filed Mar. 18, 2020, which is hereby incorporated by reference herein in its entirety.

### TECHNICAL FIELD

The present disclosure relates generally to the field of identifying healthcare service and supply eligibility for an insured and/or an insured's dependent(s), and related costs, discounts, and/or subsidies, and more particularly to systems and methods for identifying service and product eligibility across multiple platforms.

### BRIEF DESCRIPTION OF THE DRAWINGS

The present embodiments will become more fully apparent from the following description and appended claims, taken in conjunction with the accompanying drawings. Understanding that the accompanying drawings depict only typical embodiments, and are, therefore, not to be considered limiting of the scope of the disclosure, the embodiments will be described and explained with specificity and detail in reference to the accompanying drawings.

FIG. 1 is a system topology diagram for a provider partner interface system (“PPIS”), according to an embodiment of the present disclosure.

FIG. 2A is a diagram of a PPIS, according to one embodiment, launching multiple ephemeral containers to handle eligibility and benefits requests.

FIG. 2B is another diagram of the PPIS of FIG. 2A, illustrating an architecture according to one embodiment.

FIG. 3 is a relational diagram of a PPIS, according to one embodiment.

FIG. 4 is an illustration of a BMP administration screen of a BSP for a PPIS, according to an embodiment of the present disclosure.

FIG. 5A is an illustration of a search input screen of a PPIS, according to an embodiment of the present disclosure.

FIG. 5B is an illustration of the search input screen of the PPIS of FIG. 5A, and the results region also shows a recent search result set for a previous search requested by the current user identified by the username.

FIG. 5C is an illustration of the search entry screen of the PPIS of FIGS. 5A and 5B, showing a completed search based on the input of FIGS. 5A and 5B.

FIG. 5D is a search entry screen with a benefits eligibility overview screen for the PPIS of FIGS. 5A-5C.

FIG. 5E is an illustration of a plan snapshot screen of the PPIS of FIGS. 5A-5D.

FIG. 5F is an illustration of the plan snapshot screen of FIG. 5E, with the plan benefits tab selected.

FIG. 6A is an illustration of a copay calculator screen of a PPIS, according to an embodiment of the present disclosure.

FIG. 6B is an illustration of a copay calculator review/checkout screen of the PPIS of FIG. 6A.

FIG. 7 is an illustration of a search entry screen of a PPIS, according to one embodiment, and showing benefit information from more than one of the plurality of BMPs.

FIG. 8 is a method diagram for a PPIS, according to an embodiment of the present disclosure.

FIG. 9 is an error handling method diagram for a PPIS, according to an embodiment of the present disclosure.

FIG. 10 is a copay calculator method diagram for a PPIS according to one embodiment.

FIG. 11 is a relational diagram of a PPIS, according to an embodiment of the present disclosure, and illustrates creation (and maintenance) of a master list and a logic library.

FIG. 12 is a relational diagram of a PPIS , according to an embodiment of the present disclosure, and illustrates eligibility assessment, according to an embodiment of the present disclosure.

### DETAILED DESCRIPTION

The present disclosure is directed to systems and methods for discovering and/or identifying healthcare services, supplies and/or product eligibilities for an insured (e.g., a patient) and their dependents, and related costs, discounts, and/or subsidies (e.g. the insured's co-pay amount (or discount or coverage benefit) for an examination, contact lenses, eyeglass frames, and eyeglass lenses). The present disclosure is also directed more particularly to systems and methods for identifying the service eligibility across multiple insurers (e.g., potentially an unlimited number of insurer), multiple benefits plans, services, products, platforms and/or providers, for an insured and their dependents. An insured or a healthcare provider can generate a bundle of services, supplies and/or products that are consumed or are to be consumed, and which may be associated with a particular visit and/or treatment and supplied and/or sold by the healthcare provider. The bundle of services, supplies, and/or products may be selected from or may be pre-associated with a preconfigured inventory of services, supplies and products, each with an associated cost or pricing (scheme), that the healthcare provider may regularly provide to patients in the course of operation. Potentially an unlimited number of insurers, benefits plans, benefits, and/or related business rules and platforms can be considered by the healthcare provider or insured(s), in order to determine the patient's present eligibilities and to calculate pricing for the patient's visit or treatment, given their discounts or subsidies (copay).

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the disclosure, as claimed, but is merely representative of various embodiments. While the various aspects of the embodiments are presented in drawings, the drawings are not necessarily drawn to scale unless specifically indicated.

Many individuals are eligible for a variety of benefits, according to one or more benefits plans. Benefits plans may be insurance plans provided by an insurance company, or additional benefits provided by an entity on behalf of an insurance company. The individuals may obtain eligibility according to one or more benefits plan(s) through enrollment in such benefits plan(s), such as by virtue of insurance programs that may provide health-related services plans (e.g., an optical or vision care plan, a dental plan, a health insurance plan, etc.). A Benefit Service Provider (“BSP”) may request benefit information from a Benefit Management Provider (“BMP”) upon request from an insured in conjunction with a request for or about the insured (or dependent) receiving care from the BSP. Similarly, an insured may request benefit information from a BMP. A BMP may work with an insuring entity (“insurer”) to provide these benefit management services, while in some instances, the BMP may be the insurer. In many instances, an individual (a beneficiary) who is eligible for one or more benefits under a benefits plan (e.g., a health-related services plan) contacts a BSP and provides personally identifying information. The BSP, at some point prior to, during, or after providing the benefit(s), contacts a BMP to verify eligibility of the beneficiary for the benefit(s); and to ascertain details, such as procedures, services, products, appliances, etc., authorized by the beneficiary's benefits plan(s), as well as information about the financial provisioning for the benefit(s) (amount(s) payable to the BSP by the plan, copays payable by the individual for services and products, benefits expiration and renewal dates and/or service date ranges by treatment and/or product type, etc.). Each BMP may store benefit and beneficiary information in different formats and using different systems and/or platforms.

It is not uncommon for the beneficiary seeking the benefit(s) to be covered by multiple benefit plans. For example, coverage by multiple benefit plans may arise in a situation where the individual's employer provides an insurance plan comprising a health-related services plan (as a primary-insured), and the individual is covered by another plan (as a dependent-beneficiary), for example, by virtue of relationship to another individual having such coverage, such as in a spousal relationship, or when an individual who is both a dependent (for insurance purposes) of an insured and is also an insured as a primary member under his/her own plan. Such an individual may be unaware that he/she may be a beneficiary under multiple benefits plans.

Commonly, a licensed practitioner works at, or is a principal of a company (e.g., a “practice”) where provisioning (e.g., providing a service such as an examination or providing a device such as eyeglasses) may be performed. The practice may have a number of practitioners associated, and generally has a number of non-practitioner employees (receptionists, aides, assistants, pre-licensing affiliates, accounting personnel, etc.) Relatedly, a practitioner may be affiliated with multiple practices, or with a practice having multiple locations. Due to business rules and as a means of furthering compliance with the Health Insurance Portability and Accountability Act of 1996 (“HIPAA”), a BMP may provide access to benefit information to a licensed practitioner at a BSP, in a particular field, such as an ophthalmologist, an optometrist, a dentist, an oral surgeon, etc., but not to other persons affiliated with the practitioner. Thus, the practitioner finds him-/herself needing to share the theoretically-unique credentials for obtaining information from each BMP with other persons, such as a staff member employed at the BSP.

It is not uncommon for an individual to seek provisioning from a BSP while being uncertain of the source of a pertinent health-related services plan. For example, an employment transition may raise a question about which BMP the BSP should be contacting, or an individual may have lost the information from a plan provider. Another example from the arena of vision care is that often a BMP that focuses on providing medical insurance may contract out dental and/or vision insurance (sometimes known as supplemental insurance) as part of an overall plan of the BMP, but does not issue the supplemental insurance cards (that would allow a BSP to identify the BMP) for the dental and/or vision portion of the overall plan. Furthermore, due to many variations between health-related services plans, variations within a plan, as well as variation as to the business logic required to determine eligibility for a given (bundle of) service(s), supply(/-ies) or product(s), and to calculate the copay amount and/or fees to charge the patient, a BSP may not have a reliable and repeatable means of automatically determining provisioning available to a particular individual, or to determine costs to be borne by the individual. In view of the potential breadth of such variations, neither the patient (or insured) nor the BSP can readily, easily, and quickly identify a BMP or an insurer for the insured and/or dependent(s), available benefits plans, related eligibility, and costs for treatments without individually studying, potentially, a variety of benefits plans, comparing the information against a B SP's available inventory of services, often requiring specialized knowledge of language used in a benefits plan and/or the BSP's inventory of services, and manually performing multiple series of calculations. Indeed, in an instance wherein the patient does not know the insurer or BMP, the BSP may need to resort to accessing all BMP benefits portals (websites) to research a plethora of possible benefits plans under which the patient may have coverage can be a considerably time-consuming and error-fraught exercise, often resulting in the BSP or patient stopping with the first benefits plan providing any degree of benefit for the patient, without searching for an optimal benefits approach to care, treatment, or products.

The foregoing describes a complicated, if not convoluted, landscape for patients in seeking to use benefits, for BSPs in provisioning effectively and efficiently for patients, and, albeit to a lesser degree, for BMPs in providing benefit information to individuals and BSPs. Presently, a patient or an employee of a BSP, typically, may contact at least one BMP to ascertain information about benefits available to a patient of the BSP. Upon finding the patient has benefits available through a particular BMP, the patient and/or BSP may fail to contact remaining BMPs and, therefore, not identify additional or additive benefits, nor any other benefits plan(s) (in instances where a patient is a beneficiary of multiple benefits plans) that offers the most optimal benefit for a given patient's visit, care, or procedure. Furthermore, the details of the benefits, in practical application, are often manually computed/calculated, often leading to errors in amounts claimed against the BMP or collected from the patient, or both.

The present disclosure is directed to systems and methods enabling a BSP to automatically and consistently obtain benefit information for a patient by querying a plurality of BMPs, whereby the BSP is able to (a) accurately identify benefits available to the patient, and (b) accurately correlate provisioning of such benefits, along with correct claims and patient copays.

The term coverage program administrator (also referred to herein as a “CPA”) refers to a business entity that provides insurance services or insurance-like services. An individual may be an enrollee of a benefits plan administered by a CPA. A CPA may offer benefits plans related to a particular industry or activity. For example, a CPA may offer subscription-like service into which an enrollee pays whereby the enrollee gains access to services, potentially for free at the time of service, at a discounted price, on a predefined periodicity, etc. As another example, a CPA may offer benefits plans that supplement health insurance programs, such as dental care, eye/vision care, orthopedic/orthotic services, etc.

The term benefits management provider (also referred to herein as a “BMP”) refers to a CPA that manages health-related services plans, such as, e.g., a vision care benefits manager, a dental care benefits manager, etc. A benefits management provider may be an insurer, or an entity providing at least some services on behalf of an insurer for an insured (and dependents of the insured). A benefits management provider may, on request, provide benefits information regarding an individual (e.g., an enrollee, an insured, etc.) to a benefits service provider.

The terms “enrollee,” “insured,” “primary,” “primary insured,” “dependent-insured,” “dependent,” and “beneficiary,” as used herein, refer to an individual who may be entitled to receive benefits under a benefits plan administered by a BMP. Primary insured, in particular, refers to an individual who applies for or otherwise obtains the benefits plan and, typically, whose exit from the benefits plan inherently removes any dependents enrolled in the benefits plan. In general, a benefits plan has exactly one primary insured.

The term benefit service provider (also referred to herein as a “BSP”) refers to a business entity that provides services, supplies, and/or products that may be subject to benefits under a benefits plan administered by a CPA. For example, a BMP may offer one or more benefits plans for health-related services, such as vision care, dental care, orthodontic care, etc. A BSP operating in a health-related space typically includes at least one professional practitioner licensed in the relevant health-related care field, such as, e.g., an ophthalmologist, an optometrist, a dentist, an oral surgeon, an orthodontist, etc. A professional practitioner may be a principal in the business, a partner, an affiliate, an associate, an employee, etc. A BSP typically has other personnel to provide supporting services, such as reception, triage, accounting, billing, assistant care providers, etc.

The phrase “plan administration,” as well as similar phrases, e.g., “administer a plan,” refers to functions performed by a BMP including, but not limited to, enrolling insureds in a benefits plan whereby the insureds may obtain benefits established within the benefits plan, processing claims against the plan for services performed by a BSP for or on behalf of an insured, making payments to the BSP in accordance with terms of the benefits plan for services performed and/or products supplied on behalf of an insured of the plan, etc. Administering a plan may comprise the BMP providing benefits plan details to a BSP preparatory to or in conjunction with the BSP providing services and/or products to an insured.

The term “benefit” as used herein refers to a transactional obligation (or a contribution) on the part of an insurer (that may be represented by a CPA or BMP) toward an insured or a dependent of an insured with regard to a service or product, or combination of one or more services and/or one or more products. A service may be a tangible or intangible product (e.g., an examination, inspection, fitting, adjustment, installation, removal, etc.), while a product may generally be a tangible good (e.g., eyeglasses, contact lenses, frames, orthodontic appliance, brace, etc.)

The terms “provision” and “provisioning” as used herein refer to providing a service or product, or a combination of service(s) and/or product(s) by a B SP to an insured or a dependent of an insured, and may include providing such service(s)/product(s) when a reasonable expectation exists that the insured or dependent is entitled to a corresponding benefit.

The terms “benefit” and “provision”/“provisioning” may simultaneously refer to the same in se service(s)/product(s) and may be distinguished by the nature of the relationship between a patient and an involved entity. More particular, the term “benefit” may be applicable wherein the relationship lies between a patient and a BMP; while the terms “provision” and “provisioning” may be applicable wherein the relationship lies between a patient and a BSP. By way of non-limiting example, a patient may have a benefit from a BMP for a service/product to be provisioned by a BSP.

While the present invention may apply to CPAs/BMPs and BSPs in a variety of industries, for convenience of the present disclosure, an embodiment applicable to a health care-related industry, vision care, is described; however, the principles underlying the present disclosure are not limited to a health care-related industry, field, CPA, BMP, or BSP. The present disclosure anticipates embodiments not expressly discussed herein, and in a variety of industries and areas of service.

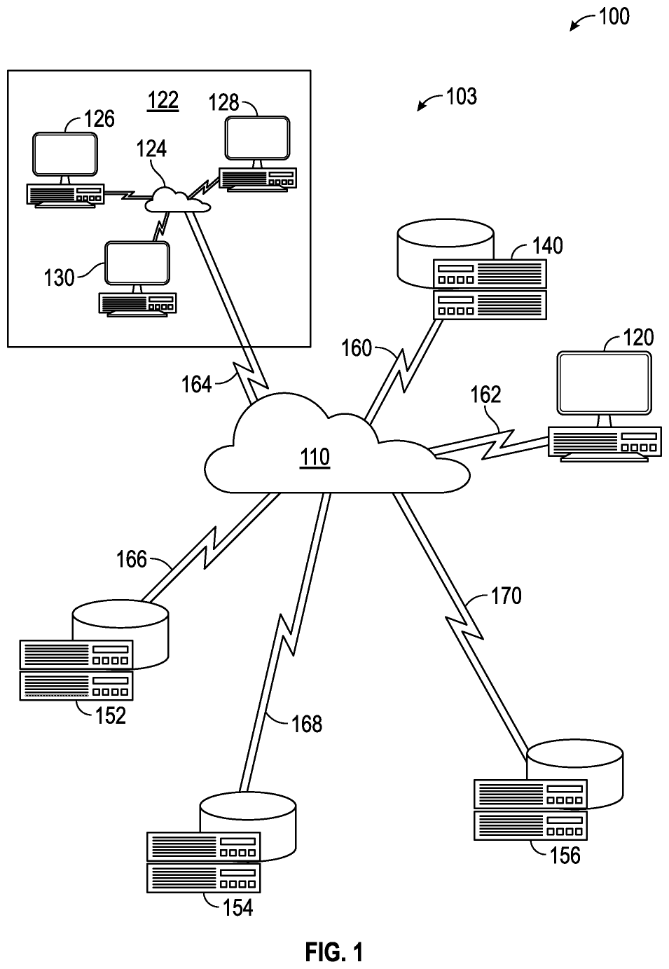

FIG. 1 is a system topology diagram 103 for a provider partner interface system (“PPIS”) 100, according to an embodiment of the present disclosure. The PPIS 100 comprises a PPIS server 140 that may be interoperable with or accessible to at least one benefits service provider (“BSP”) computing system 120,122 (e.g., an electronic health record (“EHR”) system or patient insurance benefits portal of a vision care provider (“VCP”) or a medical care provider (“MCP”)) 120, 122. The PPIS server 140 may also be optionally interoperable with or able to communicate over a network with one or more benefits management provider (“BMP”) computing systems 152, 154, 156. A BMP computing system 152, 154, 156 or BSP computing system 120, 122 may comprise an enterprise-level server, a virtual or cloud server, or a web-based or Internet portal system (“web server”), or any other computing systems known in the art. Reference is made throughout the disclosure to a “BMP server;” however, this is merely for ease of reference and not intended to limit in any way a type of computing system at a BMP with which the PPIS 100 may communicate. Accordingly, communication between the PPIS server 140 and a BMP server may use any appropriate communication protocol, such as Hyper-Text Transport Protocol (“HTTP”), a web browser, an Application Program Interface (“API”), or other methods of interoperability, etc. For the purposes of the present illustration, a smaller BSP system 120 and a larger BSP system 122 are shown. The larger BSP system 122 may have more users 126, 128, 130 than does the smaller BSP system 120. In some embodiments, the larger system BSP 122 may have an intranet 124. FIG. 1 shows three BMP servers 152, 154, 156. In some embodiments of the PPIS 100, there may be fewer or more BMP servers. The PPIS server 140, the BSP systems 120, 122, and the BMP servers 152, 154, 156 may be connected, or otherwise in communication, such as via the Internet 110 or other network. In some embodiments, the PPIS server 140, the BSP systems 120, 122, and the BMP servers 152, 154, 156 may not be directly physically connected but simply available “online,” such as through the Internet 110 or other network.

The BSP systems 120, 122, the PPIS server 140, and the BMP servers 152, 154, 156 are shown having, respective connections 162, 164, 160, 166, 168, 170 to the Internet 110. Each represented connection 162-170 may comprise multiple connections, as is known in the art. The PPIS server 140 and the BSP systems may communicate across the Internet 110 using the connections 160, 162, and 164. The PPIS 140 may use its connection 160 to communicate through the Internet 110 to each of the BMP servers 152, 154, 156 via their respective connections 166, 168, 170.

A user authenticated to a BSP system 120, 122 may use the BSP system 120, 122 to send, via the respective connection 162, 164 a request for benefits information related to an individual (e.g., a patient) through the Internet 110 to be delivered, via the connection 160, to the PPIS server 140, or enter the same 164 request for benefits information related to an individual directly into the PPIS application for transmission to the PPIS server 140. The PPIS server 140 may, via the respective connections 160, 166, 168, 170 through the Internet 110, query one or more of the BMP servers 152, 154, 156 to ascertain which, if any, of the BMPs 152, 154, 156 has benefits information for the particular patient. For a BMP server comprising a “web portal,” queries may provide particularized inputs (e.g., programmatic inputs, such as by a bot) to the web portal to request output (in the form of structured web page) comprising benefits information related to a particular benefits plan or an individual (patient, insured, dependent, etc.) who may be a beneficiary of under any benefits plan of the BMP 152, 154, 156. None, one, or a plurality of the BMPs 152, 154, 156 may have benefits information for the particular patient. The PPIS server 140 and each BMP 152, 154, 156 may communicate across the Internet 110 using the connections 160, 166, 168, 170 to request and capture any benefits information for the particular patient that each BMP 152, 154, 156 has with the PPIS server 140. The PPIS server 140 may aggregate any information received about the particular patient from the respective BMPs 152, 154, 156, and return the same to the requesting BSP 120.

A user (e.g., a practitioner or an assistant) at a BSP system 120 may interface with the PPIS 100 and/or otherwise utilize the PPIS 100 to identify eligibility of a patient for a benefit (e.g., a product or service) and to capture (e.g., download, extract from a webpage etc.) the patient's benefits plan. The PPIS 100 facilitates the user identifying the eligibility benefits plan(s) of the patient across multiple BMPs by interfacing or otherwise communicating with multiple BMP servers 152, 154, 156 (as by, for example, a BMP's web portal). The PPIS 100 may facilitate generation of an aggregated set of patient eligibility data and benefits plans that can be inserted, stored and utilized by the practice providers, while simultaneously collecting and publishing the same results within the PPIS for all patients searched within a provider practice for collective review by one or more members of a provider practice (aggregated patient search results).

For example, an optometrist at a clinic may access a BSP system 120 to interface or otherwise access the PPIS 100 to identify eligibility (e.g., insurance coverage under an insurance plan) of a patient for eyeglasses, or directly access the PPIS 100 to identify the same eligibility. A patient may also be able to access the PPIS 100, such as by an Internet-connected device (e.g., a home or work computer, a web-enabled mobile phone, a computing tablet, etc.) to identify eligibility, etc. The PPIS 100 may identify insurance coverage to pay for the eyeglasses by interfacing one or more BMP servers 152, 154, 156 and identifying coverage eligibility across the one or more BMPs. In this manner, the user can readily obtain information and provide the information to the patient about how much that patient can expect to pay out of pocket for the pair of eyeglasses, for example. The PPIS 100 may identify overlapping eligibility. The PPIS 100 may calculate or otherwise determine a co-payment for the patient. The PPIS 100 may manage access credentials at the BSP system 120 such that the access credentials of an authorized user (e.g., the ophthalmologist) are maintained separate from and confidential relative to assisting personnel users (reception, triage, medical assistants, etc.). The PPIS 100 may, in one embodiment, accomplish the identifying of eligibility of a benefit across multiple platforms by launching multiple virtual servers to each provide concurrent interface with a respective BMP server 152, 154, 156, as will be explained in more detail. In one embodiment, a virtual server may not be employed.

Furthermore, in one embodiment, when a patient (or provider on behalf of a patient) intends to elect or elects to use a particular benefit under a benefits plan, in addition to other functionality described herein, the PPIS 100 may immediately communicate use (or intent to use by issuing an authorization or request for authorization) of the particular benefit to the respective BMP 152, 154, 156. In other words, use (or intent to use or request for authorization) of a benefit for a patient may be communicated to the respective BMP 152, 514, 156 in real time or near real-time. Communicating use of a particular benefit by a patient may allow the respective BMP 152, 154, 156 to update relevant benefit status, such as availability dates, remaining benefit amount, existence of and unavailability of the particular benefit (potentially for a specific period of time), authorization status etc.

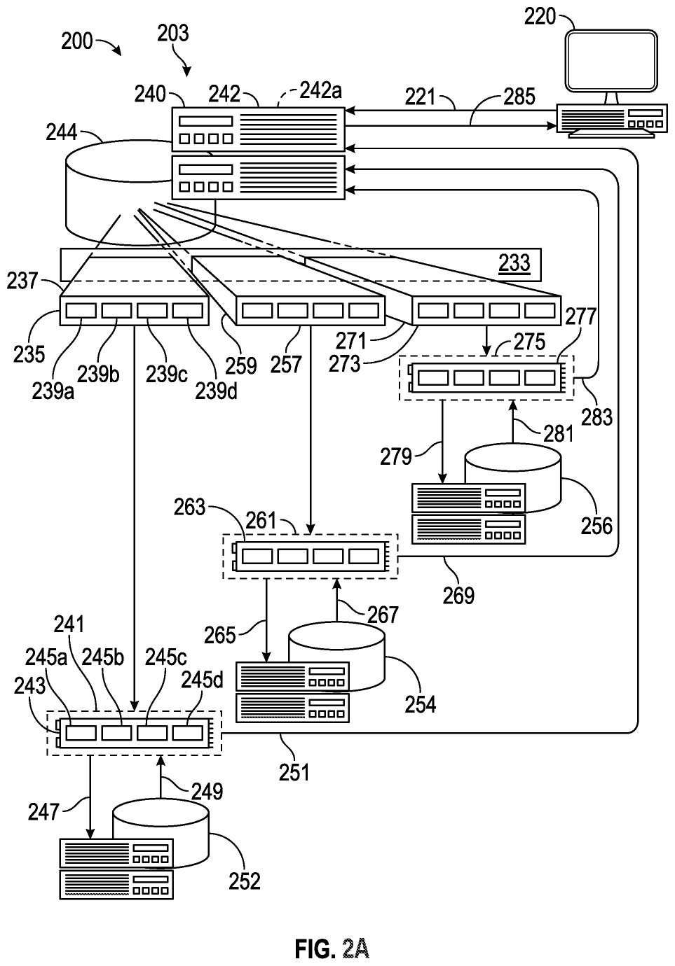

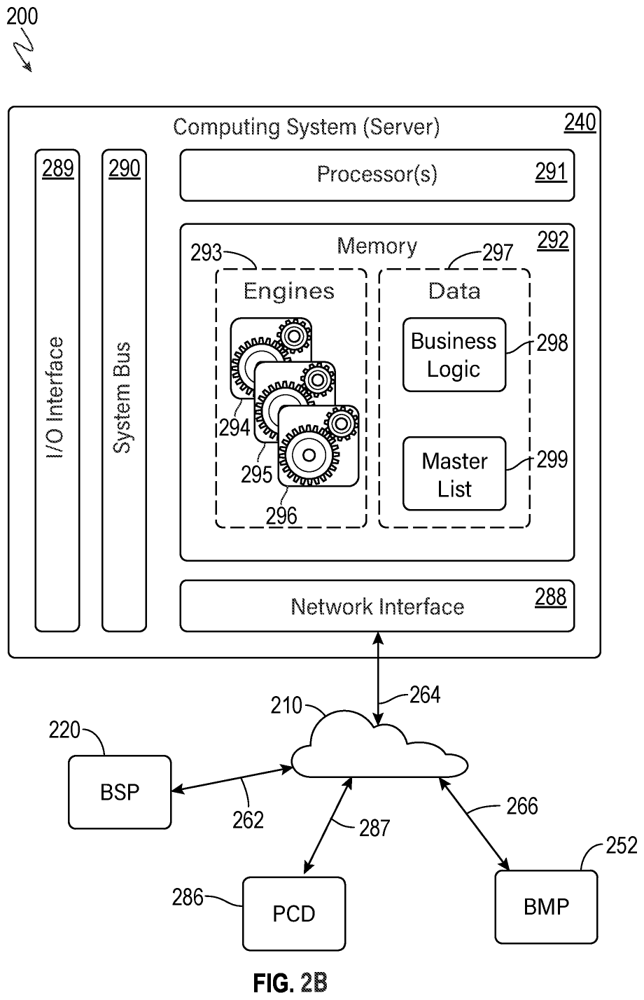

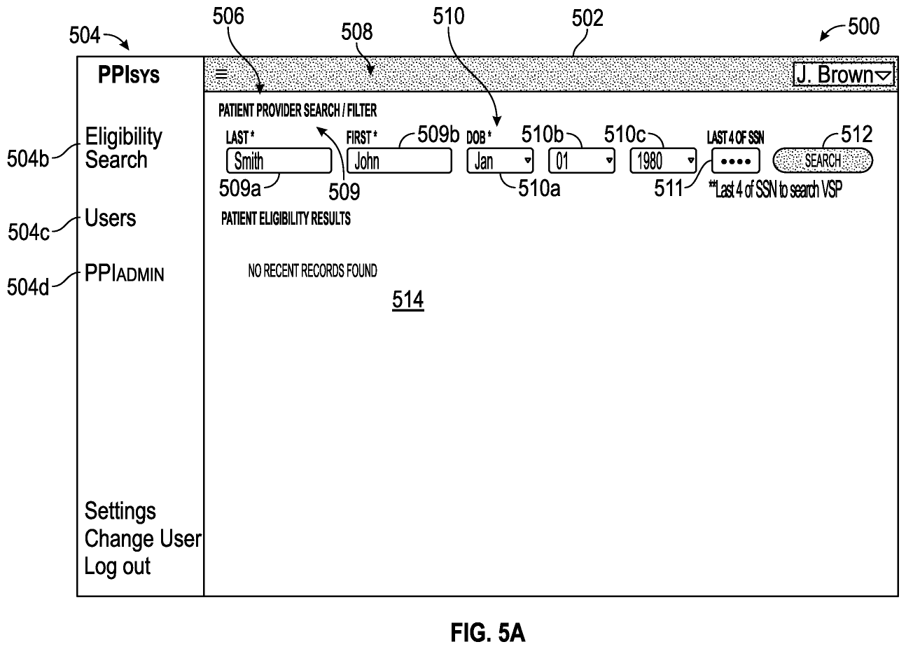

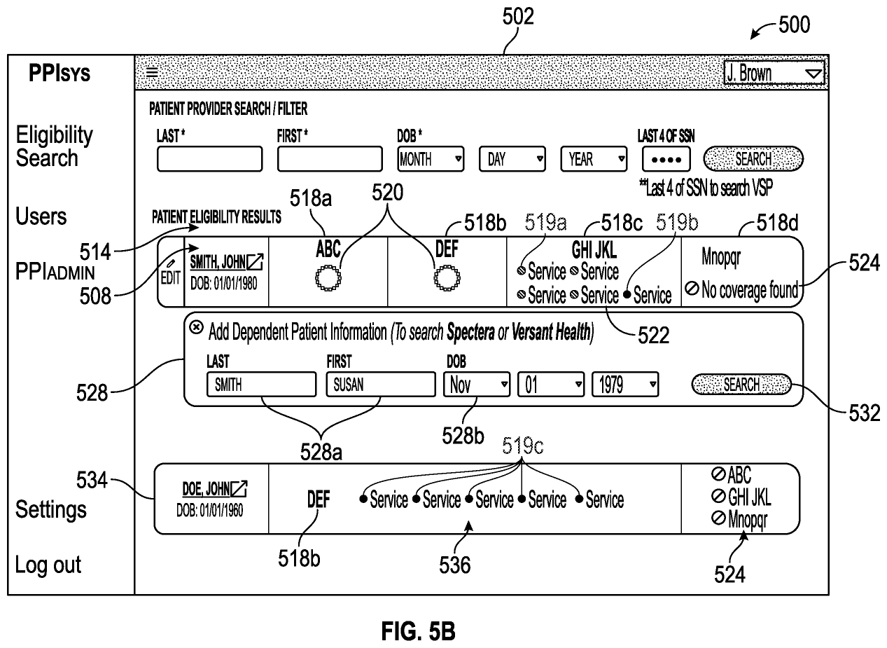

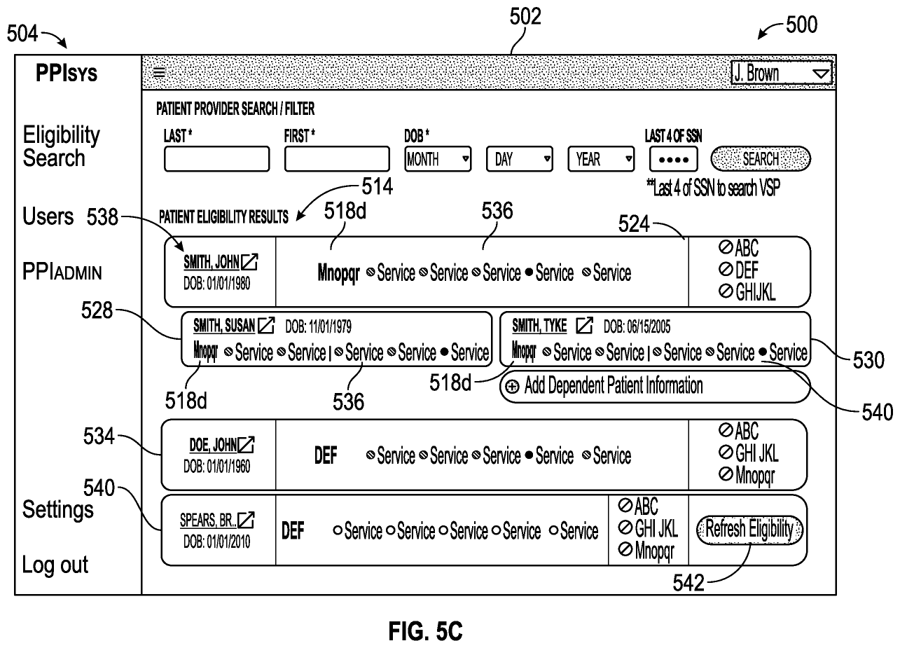

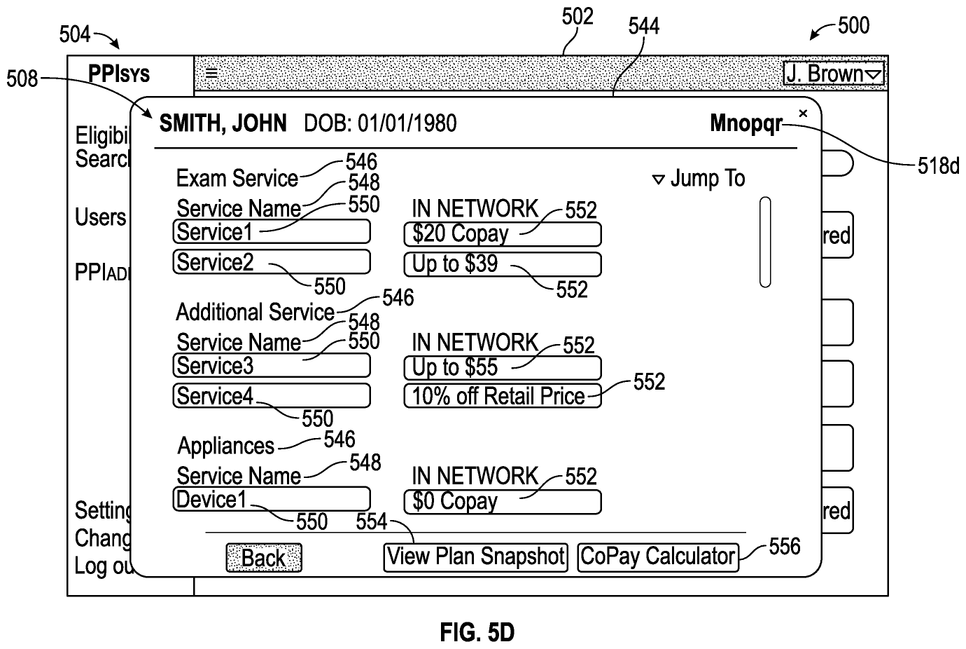

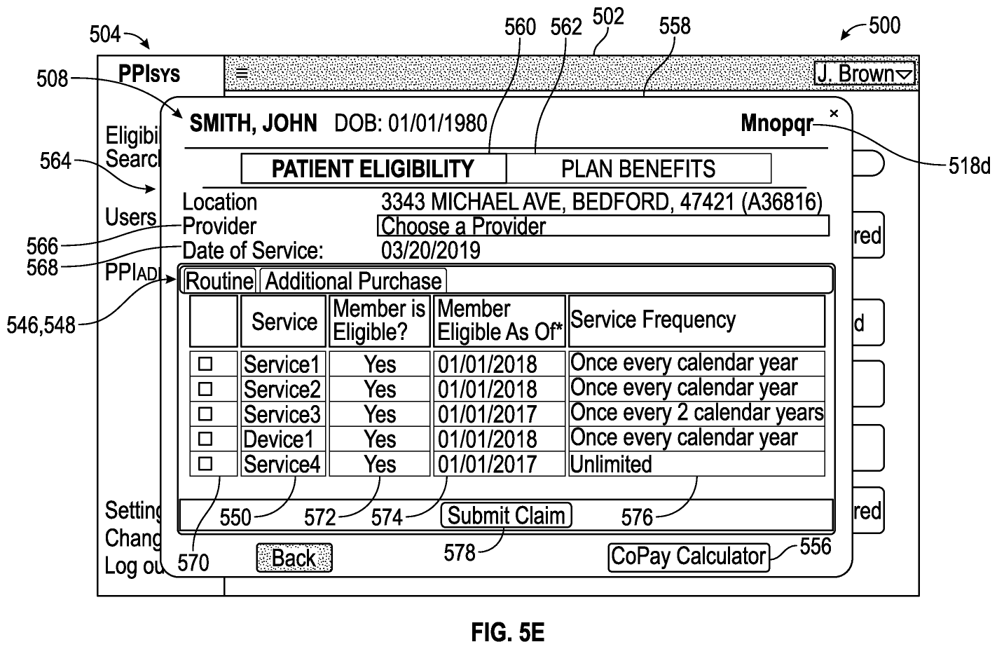

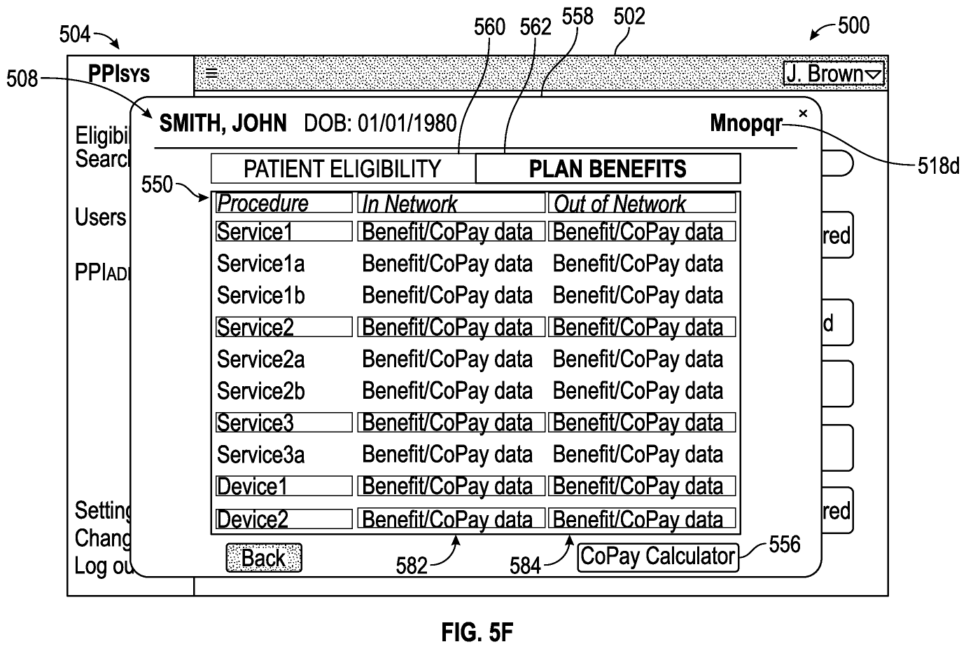

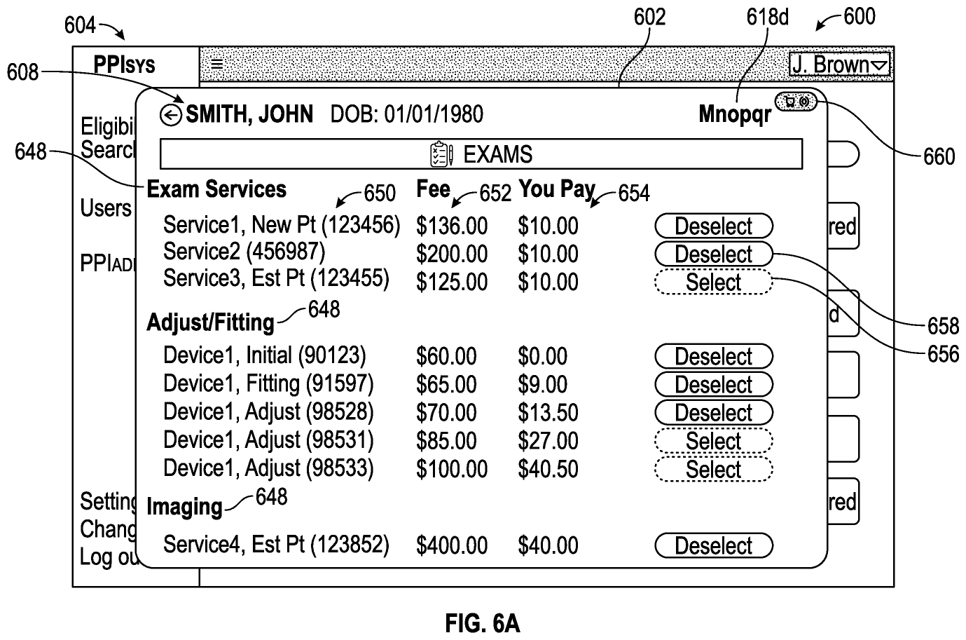

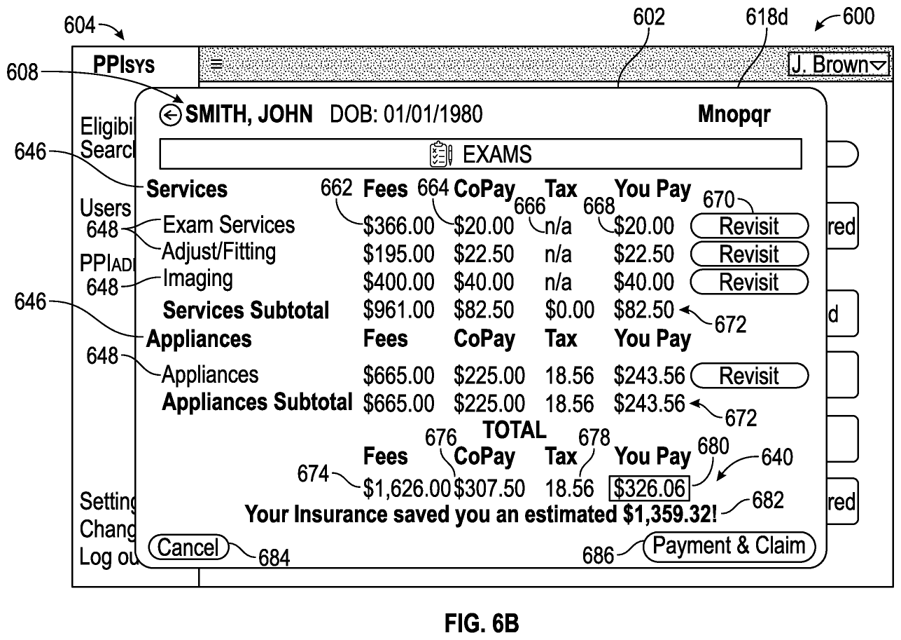

FIG. 2A depicts an embodiment of a PPIS 200. The PPIS 200 of FIG. 2A may resemble the PPIS 100 described above in certain respects. Accordingly, like features are designated with like reference numerals, with the leading digit(s) incremented to “2.” For example, the embodiment depicted in FIG. 2A includes a PPIS server 240 that may, in some respects, resemble the PPIS server 140 of FIG. 1. Relevant disclosure set forth above regarding similarly identified features thus may not be repeated hereafter. Moreover, specific features of the PPIS server 240 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the PPIS server 240 and related components depicted in FIG. 2A. Any suitable combination of the features, and variation of the same, described with respect to the PPIS server 140 and related components illustrated in FIG. 1 can be employed with the PPIS server 240 and related components of FIG. 2A, and vice versa. For features that are repeated in subsequent figures, this pattern of disclosure applies equally to further embodiments depicted in subsequent figures and described hereafter, wherein the leading digits may be further incremented; however, for features not shown or particularly described in multiple figures, this pattern of disclosure does not apply. For example, the view plan snapshot button 554 of FIG. 5D is not the same feature as the copay 654 in FIG. 6A.

FIG. 2A is a system diagram of a PPIS 200 similar, in at least many respects, to the PPIS 100 of FIG. 1, according to an embodiment of the present disclosure. In the context of describing a computing environment such as a PPIS, reference to a BSP or a BMP includes reference to a computing device and/or computing system (e.g., the BSP system 120 of FIG. 1) of the particular BSP or BMP. The PPIS server 240 comprises a server computer 242 and a storage device 244. The server computer 242 may further comprise an internal storage device 242a. The internal storage device 242a and the storage 244 each comprises at least a non-transitory storage medium capable of electrically storing machine-executable instructions, whereby the functions of the PPIS 200 may be performed.

When a patient requests provisioning from the BSP 220, the BSP 220 may acquire from the patient identifying information for the patient, the insured and/or dependents that may be used to obtain benefits information from any of a plurality of BMPs 252, 254, 256. In other words, the PPIS 200 may acquire benefits plan data from a plurality of BMPs 252, 254, 256. The benefits plan data may include one or more benefits plans of each of the plurality of BMPs 252, 254, 256. Each benefits plan may indicate one or more benefits covered by the benefits plan and indicate a manner of determining a level of coverage for each of the one or more benefits. The BSP 220 may, via a web browser, connect 221, 285 to the PPIS server 240 to initiate a search for benefits information regarding the patient. The PPIS server 240 may reply with a browser-readable web page comprising a container or form, the web page and container configured to allow manual input of information by a user at the BSP 220 and to capture additional information that may be used to correlate the particular search request with any data subsequently retrieved from one or more of the plurality of BMPs 252, 254, 256, and may comprise data for dependents of a given primary, or for the primary of a given dependent. See FIG. 5A for an example of manual entry of data.

Data that may be automatically captured may comprise an identifier for the particular request at the BSP 220. In one embodiment, an identifier related to the particular BSP 220 may be captured, and the identifier may be used by the PPIS server 240 to retrieve credentials assigned by each BMP 252, 254, 256 for use by the particular BSP 220 in connecting with each individual BMP 252, 254, 256. In one embodiment, the BSP 220 may locally store its credentials, and the browser and container may be configured to receive the credentials from the local store at the BSP 220 and forward them to the PPIS server 240. In one embodiment, when the BSP 220 connects to the PPIS server 240 for the purpose of initiating a benefits information request, the PPIS server 240 may generate a unique query token (e.g., a “query ID”) for use throughout the particular request/response process to associate the particular request, the BSP 220 and BSP user (discussed below), and any information retrieved related to the patient who is the subject of the request. The PPIS server 240 may send the query ID, which is unique to each benefits information request, to the BSP 220. The BSP 220 may include the unique query token when providing the request for benefits information to the PPIS server 240.

The PPIS server 240 may parse the request container received from the BSP 220 to acquire the patient identifying information, the BSP credentials for connecting to each BMP 252, 254, 256, and, if previously generated, the unique query token whereby the request and all related information may be correlated. If the unique query token was not previously generated, one may now be generated to ensure that all responses from the various BMPs 252, 254, 256 are properly related back to the original request for benefits information. The request, along with the unique token, may be placed in a logical queue of the PPIS server 240. The parsed request may be passed to an ephemeral container controller 233. The ephemeral container controller 233 may retrieve 237, 259, 271 from the PPIS server 240 a set of master container templates (“MCT,” “MCTs”) 235, 259, 273. Each MCT 235, 259, 273 corresponds to exactly one BMP 252, 254, 256. For the present example, the first MCT 235 corresponds to the first BMP 252, the second MCT 259 to the second BMP 254, and the third MCT 273 to the third BMP 256. Each MCT 235, 259, 273 contains information related to its corresponding BMP 252, 254, 256.

Each BMP 252, 254, 256 may employ a different web portal configuration, computer server operating system, and/or each may use a different database package or a differently configured database system. For example, the BMP 252 may use Canonical® Ubuntu® as its server operating system (“OS”) and may be running an Oracle® database; the BMP 254 may use the IBM® z/OS® server OS running IBM® DB2® database services. BMP 256 may use IBM z/OS server OS to run an Oracle database. Furthermore, although BMP 252 and 256 may both use an Oracle database, the particular installation, version, and organizational structure of one is almost certainly different from the other, and thus query formation for each BMP 252, 254, 256 will vary, as will any response. Similarly, different server OSes for each BMP 252, 254, 256 may necessitate variations in the way in which the PPIS server 240 interacts with each BMP 252, 254, 256. In addition to variations in computing environments, each BMP 252, 254, 256 may employ different design and hypertext markup (HTML), plan format and formatting, plan contents and structure, benefits related-business rules, formula(-ae), and/or logic. Each BMP 252, 254, 256 may employ lexicologically diverse descriptors even for essentially similar benefits plans or benefits within a benefits plan. Each MCT 235, 259, 273 may be particularly configured for a corresponding BMP 252, 254, 256. Thus, in one embodiment, the MCT 235 may contain particular server communication instructions related to the Canonical Ubuntu server OS of BMP 252, and particular database query language related to the particular installation, configuration, and organization of the Oracle database at BMP 252. The MCT 259 may be similarly configured for the OS and database at BMP 254, and the MCT 273 for the OS and database at BMP 256. The set of OS-related instructions and database-related instructions define an instruction set. In another embodiment, the MCT 235 may contain one or more structured web pages to facilitate querying a web portal at a particular BMP 252, 254, 256 based on the particular configuration of the web portal, and response containers to capture data provided by the web portal. An instruction set 239c is shown for the MCT 235. The MCT 235 may further be configured with a placeholder for patient identifying information 239a, a placeholder for a query ID 239b, and a results container 239d. Because of differences in the various BMPs 252, 254, 256, results received from them may be in different forms or may be provided with different nomenclature identifying various data, hence each results container may be configured to receive the results based on the different forms of the BMPs 252, 254, 256. The MCTs 259, 273 may be similarly configured with placeholders for patient identifying information and unique query token, an instruction set, and results container.

The ephemeral container controller 233 may create an ephemeral container 243 based on the contents of the MCT 235, and may insert into the ephemeral container 243 the patient identifying information 245a from the request in place of the placeholder 239a, and the query ID 245b in place of the placeholder 239b, a copy of the instruction set 245c from the instruction set 239c, and a copy of the results container 245d from the results container 239d. Hereafter, reference to the instruction set 245c refers to an instruction set of any ephemeral container unless particularly denoted otherwise. The ephemeral container controller 233 may also generate a corresponding ephemeral container 263 from the MCT 257 for the BMP 254, and an ephemeral container 277 from the MCT 273 for the BMP 256. Each ephemeral container 243, 263, 277 may further be configured to self-destruct once its instruction set has been executed and results have been returned to the PPIS server 240.

In one embodiment, the ephemeral container manager 233 may request the PPIS server 240 to generate a virtual server 241 into which the ephemeral container 243 may be inserted. In one embodiment, the ephemeral container manager 233 may forego the request to generate a virtual server 241; and, in such an embodiment, the functionality herein ascribed to or associated with the virtual server 241 may be accomplished by interaction between the ephemeral container 243 and the PPI server 240. In one embodiment, the ephemeral container manager 233 may be configured to create the virtual server instance 241. A virtual server 261 may also be created for the ephemeral container 263, and a virtual server 275 may be created for the ephemeral container 277. Furthermore, each MTC 235, 259, 271, and, hence, each ephemeral container 243, 263, 275 may contain within the instruction set 245c logic enabling the PPIS 200 to perform searches to identify dependents of the particular patient, and, further, to identify a primary insured in an instance wherein the patient is a dependent.

In one embodiment, the ephemeral container manager 233 may insert patient identifying information as a distinct payload within the ephemeral container, such as is shown with the patient identifying information 245a and the ephemeral container 243. In one embodiment, the ephemeral container manager 233 may modify the instruction set, such as the instruction set 245c of the ephemeral container 243 such that the patient identifying information is inserted into relevant portions of the instruction set. In one embodiment, a mix of these two methods (and other methods) of placing patient identifying information may be applied by the ephemeral container manager 233.

The virtual server 241 parses the ephemeral container 243 and may execute the instructions of the instruction set 245c. In an embodiment wherein the patient identifying information 245a is a distinct payload within the ephemeral container 243, the virtual server 241 may recompose the database queries of the instruction set 245a by appropriately applying the appropriate patient identifying information. In other words, the patient identifying information 245a indicates the patient's name is John Doe, and if a query instruction (e.g., “select {phName1}, {phName2} from table PatientNames;”) is present, the virtual server 241 may recompose the query to “select ‘Doe’, ‘John’ from table_PatientNames;”. The virtual server 241 also manages communicating with the BMP 252, including connecting, negotiating, and authenticating access to the BMP 252 using the credentials of the BSP 220; connecting with the database of the BMP 252, passing queries to the database, receiving responses from the BMP 252, formatting and placing response information into the response container 245d; transferring the response container 245d back to the PPIS 240; and identifying errors and notifying the PPIS server 240 of the error(s).

Additionally, as each virtual server 241, 263, 275 executes the logic in the instruction set 245c directed toward identifying dependents and primary insureds, when a BMP 252, 254, 256 indicates the patient does have a beneficiary within the patient's health-related service plan, if the patient is a dependent of a primary insured then additional logic may be executed to retrieve identifying information and benefit information for each dependent and/or primary insured. In one embodiment wherein the BMP 252 has provided information to the virtual server 241 about the existence of a dependent or primary insured related to the patient, the instruction set 245c of the ephemeral container 243 may comprise additional logic whereby the virtual server 241 runs additional queries against the database of the BMP 252 to retrieve the identifying information and benefits eligibility for each identifiable dependent or primary insured related to the patient. The virtual server 241 may also modify the results container 239d to accommodate the added data. In one embodiment, the instruction set 245c of the ephemeral container 245 may contain logic whereby the virtual server 241 may ascertain that a dependent or primary insured related to the patient exists. If a result indicates a dependent of a primary insured exists, the additional logic may cause the virtual server 241 to notify the PPIS server 240 to initiate a subsequent search related to the patient and targeting the one or more dependents or primary insured related to the patient. In the latter case, the instruction set 245c may further contain logic to prevent redundant recursion when the newly spawned virtual server and ephemeral container interact with the BMP 252 and determine that an individual related to the patient of the new instance exists and wherein the individual related to the patient of the new instance is the patient in the first instance, or is a dependent or primary insured identified through the first instance.

Additional virtual servers 261, 275 perform similarly with regard to their respective BMPs 254, 256. Each virtual server 241, 261, 277, as its name suggests, may be configured to self-destruct after completing transfer of the results container (or relevant error message(s)) to the PPIS server 240. One may note that the ephemeral containers 243, 263, 277 may be configured to self-destruct. However, in the event an ephemeral container 243, 263, 277 fails to self-destruct, the self-destruction of the relevant virtual server 241, 261, 275 may also accomplish destruction of the ephemeral container 243, 263, 277.

The PPIS server 240, as discussed above, uses a queue to correlate all results from BMPS 252, 254, 256 with the appropriate request from the BSP 220. Once a request is received from the BSP 220 and placed on the queue, one or more corresponding flags may be set to indicate within the queue the state of the request. For example, a first flag may indicate the request has been received at the queue, a second flag may indicate that the request has been parsed and the virtual servers 241, 261, 275 and ephemeral containers 243, 263, 277 have been dispatched, a third flag may indicate a results container 245d has been received from the virtual server 241, subsequent flags may be set upon receipt of results container from other virtual servers, a particular flag may be set for any BMP 252, 254, 256 for which an error is encountered (as described below), and a flag may be set indicating the request has been fully processed. Setting of each flag for the particular request on the queue makes possible the near-real time updating of the display browser at the BSP 220. In addition to generating the ephemeral containers 243, 263, 277, the ephemeral container controller 233 may assist in memory management for the PPIS server 240. The ephemeral container controller 233 may periodically identify and count each currently existing ephemeral container 243, 263, 277 and derive memory usage of each, as well as a total amount of memory currently in use by the plurality of ephemeral containers 243, 263, 277. The ephemeral container controller 233 may be configured with a limit to the number of ephemeral containers 243, 263, 277 that can be spawned in a given period of time, a limit to the number that may exist at any given moment, a limit to the duration each may exist, a limit to the number of each that may exist at a given time for a given BSP, a number that may be spawned in a given period for a particular BSP, etc. Furthermore, the ephemeral container controller 233 may be configured to notify a user (an end user at a BSP, or an operator of the PPIS 200) when a limit is reached. In one embodiment, the ephemeral container controller 233 may be configured to prevent generation of new ephemeral containers 243, 263, 277 when the ephemeral container controller 233 determines the current number of each has reached a particular amount (actual or percentage) of memory usage. The ephemeral container controller 233 may be configured to identify a “zombie” ephemeral container 243, 263, 277 (has ceased to perform has intended), and may further be configured to destroy an ephemeral container 243, 263, 277 (“zombie” or otherwise) and release the memory used by the now-destroyed ephemeral container 243, 263, 277 back to the system. The PPIS 200 may be configured to render to a display for an operator of the PPIS 200 a status of the ephemeral container controller 233 and information related to generation, operation, and destruction of ephemeral containers 243, 263, 277, information related to memory usage and recovery, and tools which may permit the operator to adjust, activate, deactivate, impose limits, etc., to manage the ephemeral container controller 233 and ephemeral containers 243, 263, 277.

FIG. 2B is another diagram of the PPIS 200 of FIG. 2A, illustrating an architecture according to one embodiment. FIG. 2B illustrates an architecture of a portion of the PPIS 200 for identifying service(s) and/or product(s) across a plurality of BMPs, according to an embodiment of the present disclosure. The PPIS 200 comprises a computing system (a server) 240 a BSP 220, a BMP 252, a personal computing device (“PCD”) 286, and a network (e.g., the Internet) 210. While one instance of the BSP 220, the BMP 252, and the PCD 286 are shown, this is for convenience of the disclosure and not by way of limitation. The computing system 240 includes a network interface 288 to facilitate communication between the computing system 240 and the network 210 in order to communicate with other computing systems, such as a computing system of the BSP 220, a computing system of the BMP 252, and the PCD 286. The computing system 240 further comprises an input/output interface (i/o interface) 289, a system bus 290, one or more processors 291, and an electronic memory 292. The i/o interface 289 and the system bus 290 may be configured to facilitate communication between components of the computing system 240.

The one or more processors 291 may include one or more general purpose devices, such as an Intel®, AMD®, or other standard microprocessor; and/or special purpose processing device, such as ASIC, SoC, SiP, FPGA, PAL, PLA, FPLA, PLD, or other customized or programmable device. The one or more processors 291 may perform distributed (e.g., parallel) processing to execute or otherwise implement functionalities of the present embodiments. The one or more processors 291 may run a standard operating system and perform standard operating system functions. It is recognized that any standard operating systems may be used, such as, for example, Microsoft® Windows®, Apple® MacOS®, Disk Operating System (DOS), UNIX, IRJX, Solaris® SunOS®, FreeBSD, Linux®, ffiM® OS/2® operating systems, and so forth.

The electronic memory 292 may include static RAM, dynamic RAM, flash memory, one or more flip-flops, ROM, CD-ROM, DVD, disk, tape, or magnetic, optical, or other computer storage medium, including at least one non-volatile storage medium. The electronic memory 292 is capable of storing machine-readable and -executable instructions that the one or more processors 291 are capable of reading and executing. The electronic memory 292 may include a plurality of program engines 293 and data 297, the electronic memory 292 may be local to the computing system 240 and may comprise a memory module or subsystem remote from the computing system 240 and/or distributed over a network (including the network 210)

The program engines 293 may run multiple operations concurrently or in parallel by or on the one or more processors 291. In some embodiments, portions of the disclosed modules, components, and/or facilities are embodied as executable instructions stored in hardware or in firmware, or stored on non-transitory, machine-readable storage medium. The instructions may comprise computer code that, when executed by the one or more processors 291, cause the computing system 240 to implement certain processing steps, procedures, and/or operations, as disclosed herein. The modules, components, and/or facilities disclosed herein may be implemented and/or embodied as a driver, a library, an interface, and API, FPGA configuration data, firmware (e.g., stored on an EEPROM or similar device), and/or the like. In some embodiments, portions of the modules, components, and/or facilities disclosed herein are embodied as machine components, such as general and/or application-specific devices, including, but not limited to: circuits, integrated circuits, processing components, interface components, hardware controller(s), storage controller(s), programmable hardware, FPGA(s), ASIC(s), and/or the like.

The data 297 stored on the electronic memory 425 may include data generated by the PPIS 200, such as by the program engines 293 or other modules. The data 297 may be organized as one or more data structures.

The i/o interface 289 may facilitate interfacing with one or more input devices and/or one or more output devices. The input device(s) may include a keyboard, a mouse, a touch screen, a light pen, a touch pen, a tablet, a microphone, a sensor, or other hardware with accompanying firmware and/or software. The output device(s) may include a monitor or other display, a printer, a speech or text synthesizer, a switch, a signal line, or other hardware with accompanying firmware and/or software.

The network interface 288 may be equipped with conventional network connectivity, such as, for example, Ethernet (IEEE 802.3), Token Ring (IEEE 802.5), Fiber Distributed Datalink Interface (FDDI), or Asynchronous Transfer Mode (ATM), or similar. Further, the computing system 240 may be configured to support a variety of network protocols, such as, for example, Internet Protocol (IP), Transfer Control Protocol (TCP), Network File System over UDP/TCP, Server Message Block (SMB), Microsoft® Common Internet File System (CIFS), Hypertext Transfer Protocols (HTTP, SHTTP), Direct Access File System (DAFS), File Transfer Protocol (FTP), Real-time Publish Subscribe (RTPS), Open Systems Interconnection (OIS) protocols, Simple Mail Transfer Protocol (SMTP), Secure Shell (SHH), Secure Socket Layer (SSL), and so forth.

As noted, the PPIS 200 includes various program engines 293 (or modules, etc.) to implement functionalities of the PPIS 200 and to generate, access, and/or manipulate the data 297. The program engines 293 includes one or more program engines 294 constituting the functionalities of a collector tool (further described in conjunction with the figures that follow), and on or more program engines 295 constituting the functionalities of a co-pay calculator (further described in conjunction with the figures that follow). The program engines 293 may comprise one or more additional program engines 296 to perform various functionalities of the PPIS 200.

The processor(s) 291 may be configurable to enable the processor(s) to read and execute computer-executable instructions, such as computer-executable instructions to perform the methods and/or functions described elsewhere herein. The computer-executable instructions may be stored in the electronic memory 292, or in another memory accessible to the processor(s). The i/o interface 289 and/or the network interface 288 may enable the processor(s) 291 to communicate with the electronic memory 292 and/or another memory, and to communicate with, for example, the BSP 220, the BMP 252, and/or the PCD 286. The electronic memory may store computer-readable and -executable instructions to enable the processor(s) 291 to perform the methods and/or functions described herein. More particularly, the electronic memory may store the instructions to enable the processor(s) to operate the collector tool-related engine(s) 294, the co-pay calculator-related engine(s) 295, etc. The electronic memory 291 may be configured to enable the processor(s) 291 to store, at the electronic memory 291 or at another memory, the product (data) produces by operation of the collector tool-related engine(s) 294 and the co-pay calculator-related engine(s) 295. More particularly, the electron memory 291 may be configured to store, at the data 297, business logic 298 derived by operation of the collector tool-related engine(s) 294, and a master list 299, also derive by operation of the collector tool-related engine(s) 294.

The computing system 240 may be communicatively connected 264 to the network 210, whereby the computing system 240 may be further communicatively connected 262, 266, 287 to, respectively, the BSP 220, the BMP 252, and the PCD 286. Via the communicative connections 264, 266, 258, 287, the computing system 240 may exchange, acquire, transmit, etc., data and/or instructions to, as appropriate, the BSP 220, the BMP 252, and the PCD 286.

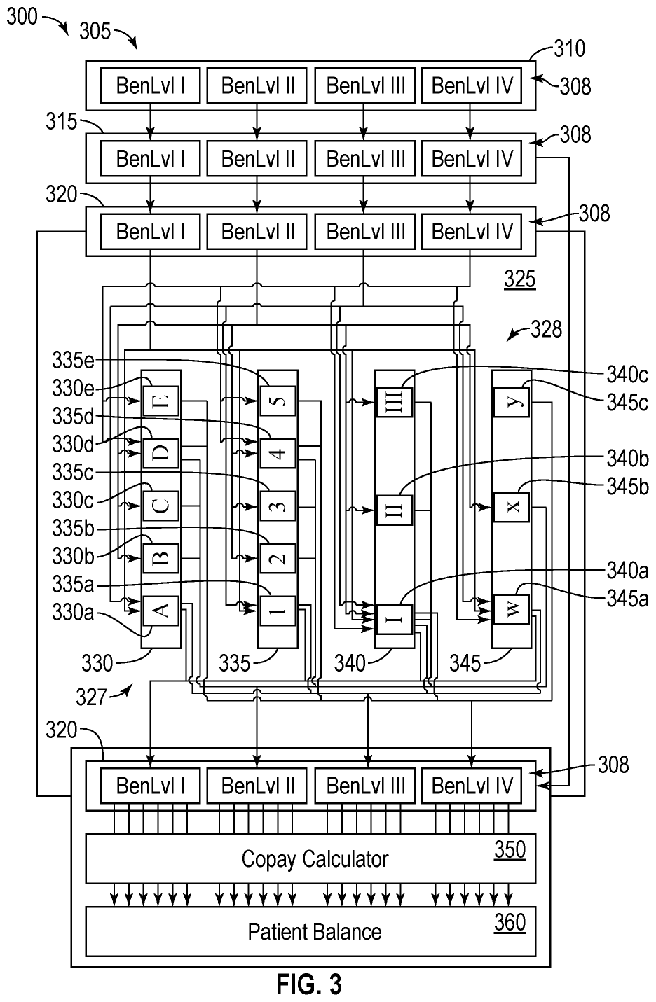

FIG. 3 is a relational diagram 305 of a PPIS 300 to determine an individual's eligibility for benefits. The PPIS 300 includes an inventory mapping tool 315, a connector tool 325, and a co-pay calculator tool 350. The co-pay calculator tool 350 may be embodied as a point of sale system that includes product browsing (shopping), a shopping cart, and checkout process to facilitate patient transactions for provider services. The PPIS 300 can acquire benefits plan data from one or more BMPs 330, 335, 340, 345 of a plurality of BMPs 327. The PPIS 300 can also acquire inventory data from a BSP 310 (whether electronically or via a provider employee through manual input or batch data loads), the inventory data including one or more inventory items (e.g., services, products) provided by the BSP 310 to individuals and associated inventory metadata (such as product and service names, descriptions, wholesale and retail prices, and related product classifications), and may generate, maintain, and/or augment a master list plan data that can be used to determine (e.g., calculate; derive a formula to calculate) an amount to be paid for a given benefit by an individual having benefits coverage (e.g., vision insurance, medical insurance, dental insurance). Each of the one or more inventory items may be a benefit covered by, or otherwise provided for under, a benefits plan of a BMP.

The connector tool 325 acquires benefits plan data from a plurality of benefits management providers (BMPs) (i.e., insurers, such as EyeMed, Davis, Spectera, etc. in a vision care context) 330, 335, 340, 345 (collectively, 327). The benefits plan data may include one or more benefits plans 330a-330e, 335a-335e, 340a-340c, 345a-345c (collectively, 328) of each of the plurality of BMPs 327. For example, each of the plurality of BMPs 327 may have its own offering of benefits plans 328 that provide varying levels of coverage for different benefits 308, such as a low coverage plan (or a low tier plan), a medium coverage plan (or a middle tier plan), a high coverage plan (or high tier plan). The levels of coverage 308 for each BMP may or may not correspond to any level of coverage for another BMP. Stated differently, it may be that no two benefits plans are alike across the plurality of BMPs 327. The benefits plan may indicate or otherwise include one or more benefits (i.e., services and/or products, such Exams, Lenses, Frames, and Contacts, in a vision care context) that are covered under the benefits plan. For example, a benefits plan may cover a benefit such as adapter lenses. (The adapter lenses may be a benefit that is a benefit class “progressive lenses” of a benefit type “lenses.”) The benefits plan may also indicate or otherwise include a level of coverage for each of the one or more benefits that are covered under the benefits plan. The benefits plan may cover all of the cost of the benefit or may only cover a portion of the cost of the benefit, according to a coverage level of the benefit under the individual's benefits plan. The benefits plan data acquired by the connector tool 325 may be utilized in at least two ways: to derive and/or generate business logic (or logic rules) and to obtain benefits information that can be mapped to inventory data of one or more BSPs. The generated business logic (or logic rules) and the benefits information mapped to inventory data can enable both determining patient eligibility for services and products and forming of calculation logic for executing calculation of discounts or copay values for products and/or services to determine personalized pricing.

As an initial phase, the PPIS 300 may acquire a plurality of plans (e.g., a sampling of plans) from each of the BMPs 327. The plurality of sample plans may comprise a sampling of actual plans in use by each BMP, a set of example plans, a combination of actual and example plans, and other plan forms. The plurality of sample plans may be provided in an electronic format, e.g., a portable document format (“PDF”) file, an encapsulated postscript (“EPS”) file, an extensible markup language (“XML”) file, a hyper-text markup language (“HTML”) file, a secure HTML (“SHTML”), a graphics interchange file (“GIF”), a Joint Photographic Experts Group (“JPG” or “JPEG”) file, a portable network graphics (“PNG”), a tagged image file format (“TIF” or “TIFF”) file, or another format electronic document. The connector tool 325 may examine or be involved with examining the plurality of plans to identify, analyze, interpret, and otherwise derive business logic and/or logic rules based on the content of the plurality of plans. More particularly, the connector tool 325 may identify the file type, then select one or more methods of acquiring human-readable text from the file.

For example, the connector tool 325 may employ regular expressions (“regex”), optical character recognition (“OCR”), or another means to read, characterize or otherwise process a file or portions of a file to identify patterns, logic, formatting, formulas, and other aspects of the file, any of which may be included in or structured into business logic and/or logic rules. In some embodiments, reading, characterizing or otherwise processing a file may include identifying visual formatting (e.g., table(s), header(s), indentation, bullet points, vertical spacing, symbols, selection indicators (check boxes, for example), text formatting (text color, background color, bolding, underlining, italics, strikethrough, etc., mathematical symbols, numbers, and so forth), sequences of characters or words, or other patterns and means whereby detailed information about the particular plan is conveyed. Reading, characterizing or otherwise processing the file may further include interpreting the acquired expressions within the file into business logic. The business logic may involve at least nomenclature directed toward eligibility, coverage, and benefits.

The business logic may act or otherwise function as a set of rules whereby the PPIS 300 will be able to determine, at a future point and from a plan for a particular individual, eligibility, coverage, and benefits, as well as characteristics of coverage and benefits. In other words, once the connector tool 325 has developed the business logic of each of the BMPs 327 for the PPIS 300, the PPIS 300 may then apply the business logic directly toward a specific plan for a particular individual to determine benefit and benefit features, such as, e.g., (in a vision care context as a non-limiting example) frequency of eye examinations, particular features of eye examinations, an insurance (BMP) contribution (or formula for calculating such), for that particular individual under that particular, individualized plan. The BMP contribution may include one or more of an amount or rate payable by the BMP toward a particular benefit for the particular individual, a discount to be applied by a BSP toward a particular benefit for the individual, a co-pay amount or rate payable by the individual to the BSP for the particular benefit, etc.

The connector tool 325 may correlate each benefit of each plan of the plurality of sample plans to one or more products or services (or combinations of products or services) of the BSP, and further develops cost calculation algorithms for each product or service (or combination thereof) of the BSP, including for products, services, or combinations not identifiable to a particular benefit of each plan. The correlated data can be used to create and/or augment a master list 320 that maps each benefit of each plan of the plurality of sample plans of each BMP to one or more corresponding products, service, or combinations thereof of the particular BSP (e.g., inventory items as may be obtained in inventory data). The master list 320 can be used to relate each product, service, or combination thereof (e.g., inventory items as may be obtained in inventory data) to every relevant benefit of each plan of each BMP. The master list 320 further provides a means of determining cost, for an individual based on a particular benefits plan of a BMP for that particular individual, a cost and cost breakdown of every service, product, or combination thereof of the BSP, including those services, products, or combination for which there may be no corresponding benefit. The cost breakdown may comprise retail price; insurance contribution in the form of payment (amount or percent-rate); limit; co-pay amount or percent-rate; tax; total for each service, product, or combination; and total for the individual.

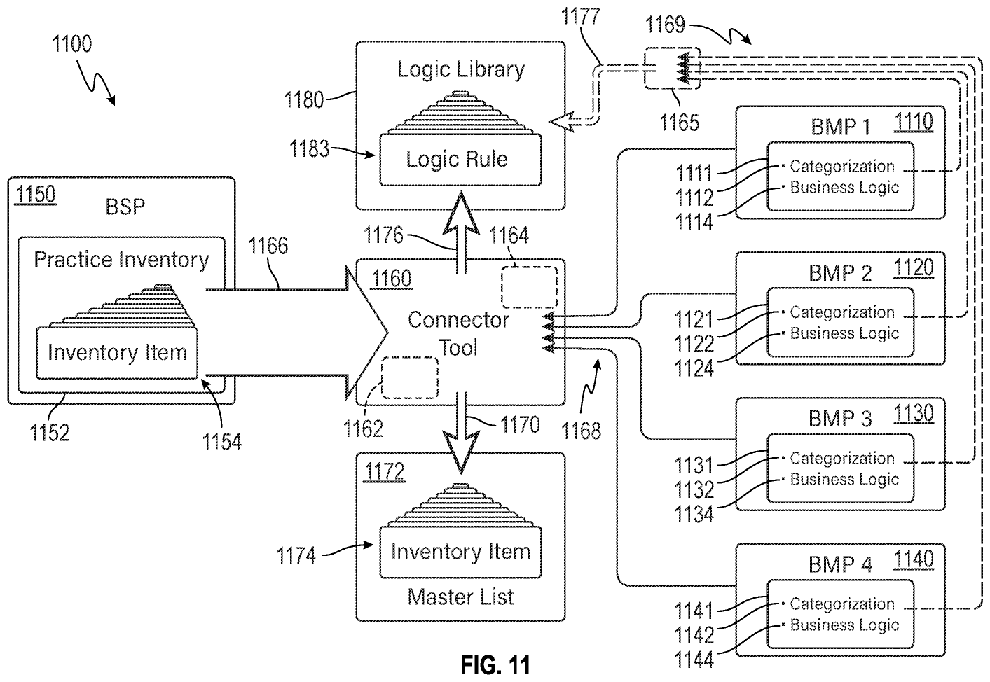

The connector tool 325 may generate and maintain a master list 320 of inventory items (e.g., services and products) of one or more BSPs 310. Each inventory item may be mapped to a benefit under one or more benefits plans administered by a BMP of the plurality of BMPs 327. The mapping may be accomplished by automatically matching product metadata and nomenclature (such as billing codes, product codes, brand and model, etc.) or other data about an inventory item and a corresponding benefit. The master list 320 is further described in conjunction with FIG. 11. The connector tool 325 may generate, maintain, and/or augment the master list 320 by identifying inventory items of BSPs 310 and correlating each to a benefit in the master list 320. The connector tool 325 provides data to the copay calculator 350. The correlation of inventory items of BSPs 310 and benefits in the master list 320 can be accomplished by identifying synonymous names (e.g., Adapter and AdapterNovo) for a given benefit that may match an inventory item of the BSP 310. The correlation of inventory items of BSPs 310 and benefits in the master list 320 can be accomplished by aid of industry codes that correspond to benefits, such as V-codes that are billing codes in vision care. For example, V code V2781 is a billing code associated with progressive lenses, such as an Adapter, and this V code can be used to correlate a given inventory item (e.g., AdapterNovo) of a BSP with a benefit (e.g., Adapter) of the master list 320.

The correlation of the inventory items 310 to the master list 320 connects or otherwise integrates the BSP into the plurality of BMPs that are likewise integrated into the master list 320. The mapping or correlation of inventory items 310 to benefits of benefits plans 328 enables enhanced capability to efficiently (and rapidly) determine a level of coverage 308 of a given benefit under a given benefits plan 328, and further to determine an amount to be paid for the given benefit by an individual having the given benefits coverage.

The inventory mapping tool 315 can acquire or otherwise receive inventory data from one or more BSPs (e.g., VCPs). The inventory data includes one or more inventory items (e.g., services and/or products) that are or can be provided by (or otherwise offered by or available from) the BSP to individuals (e.g., patients, insureds). The inventory items, in the case of a VCP (e.g., an optometrist) may include multiple benefit types, such as exams, corrective devices, etc. Within the benefits types, there may be one or more benefit classes, such as vision acuity, prophylaxis, disease remediation, lenses, frames, and contacts. The inventory items may fall within one or more of these benefit types and/or benefit classes. The inventory data may also include a price (e.g., a retail price) for each inventory item. Accordingly, if an optometrist provides or otherwise offers to patients frames for glasses, each of the frames may have an associated price within the inventory data.

The inventory mapping tool 315 may receive the inventory data from a BSP's electronic health record (EHR) system or another BSP management system. Accordingly, a BSP (e.g., an optometrist and/or clinical staff of the same) is able to simply maintain a local inventory particular to their practice and/or location. The inventory mapping tool 315 can obtain the inventory data and maintain a copy of that inventory data and that copy may be synchronized with changes made by the BSP. The synchronization can occur at regular intervals, or otherwise with appropriate or sufficient frequency to afford real-time or near-real-time accuracy of the copy of the inventory data.

The inventory mapping tool 315 can interface with the connector tool 325 to integrate new, refreshed, and/or updated inventory data of a BSP into the master list 320 to connect or otherwise integrate the BSP with the benefits plans 328 of the plurality of BMPs 327. Stated differently, the inventory mapping tool 315 can interface with the connector tool 325 to generate structured benefits plan data in the master list 320, in which each inventory item of the BSP is correlated to a benefit in the master list 320 because the benefit is covered by one or more benefits plans 328 of one or more of the plurality of BMPs 327. The structured benefits plan data of the master list 320 comprises, for each covered benefit (and, potentially for any benefit not covered by the particular plan) a benefit type (e.g., a procedure, service, appliance, etc.), an associated co-pay, limit(s), restriction(s), requirement(s), frequency, etc. The inventory mapping tool 315 connects or otherwise integrates the inventory of a BSP into the PPIS 300. Said otherwise, the PPIS 300 may determine, based on the structured benefits plan data and identifying information for an individual, that the individual is eligible for benefit coverage under a benefits plan from a plurality BMPs, and obtain a personal (to the individual) benefits plan of the individual using the identifying information.

In conjunction with a BSP providing a service for an individual, the PPIS 300 may acquire or otherwise access an electronic copy of the particular benefits plan (or benefits plans) under which the individual may be eligible for coverage, which may include multiple plans across multiple BMPs 327. The electronic copy may, for example, be a PDF, an XML file(s), an HTML file(s), an SHTML file(s), a GIF, a JPG/JPEG, a TIF, or another format electronic document or data format. The PPIS 300 examines the electronic copy to identify nomenclature (descriptions, calculation formulae, verbiage) defining benefits under the plan. The PPIS 300 can then “read” (e.g., similar to reading, characterizing, or otherwise processing the plurality of sample plans as discussed above in conjunction with FIG. 3) the individual benefits plan(s) and can employ the master list (discussed in conjunction with FIGS. 3, 11, and 12) to determine eligibility, coverage, and benefits provided under the particular plan to the particular individual.

For example, the PPIS 300 may utilize regular expressions (“regex”), optical character recognition (“OCR”), or another means to read, characterize or otherwise process the individual benefits plan(s) to identify patterns, logic, formatting, formulas, and other aspects of the file, which may be used to associate which structured business logic and/or logic rules may be appropriate for extracting relevant information from the individual benefits plan(s). In some embodiments, reading, characterizing or otherwise processing the individual benefits plan(s) may include identifying visual formatting (e.g., table(s), header(s), indentation, bullet points, vertical spacing, symbols, selection indicators (check boxes, for example), text formatting (text color, background color, bolding, underlining, italics, strikethrough, etc., mathematical symbols, numbers, and so forth) sequences of characters or words, or other patterns and means whereby detailed information about the particular plan is conveyed. Reading, characterizing or otherwise processing the individual benefits plan(s) may further include utilizing business logic to determine, from the individual benefits plan(s), the eligibility, the coverage, and the benefits, as well as characteristics of the coverage and the benefits, for the particular individual. In other words, the PPIS 300 can apply the business logic directly toward the individual benefits plan(s) to determine benefit and benefit features, such as, e.g., (in a vision care context as a non-limiting example) frequency of eye examinations, particular features of eye examinations, an insurance (BMP) contribution (or formula for calculating such), for that particular individual under the individual benefits plan(s), as well as the patient contribution or copay amount for each product and service ordered or contemplated by the patient or provider, for that patient.

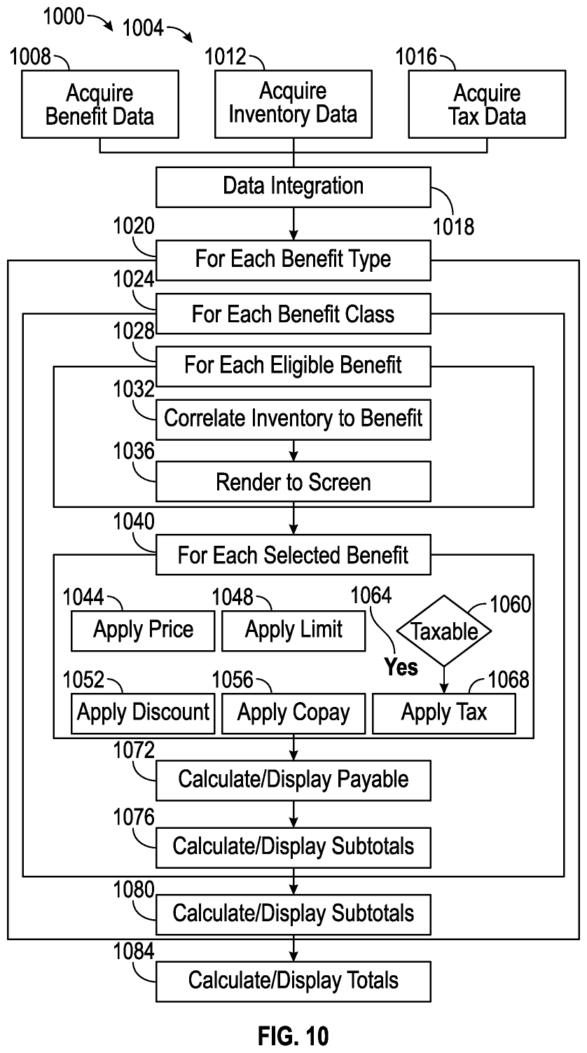

Each BMP may employ one or more formats or presentations of benefits plans, for which business logic and/or or logic rules may be used to read the benefits plan and determine information regarding each benefit of each benefits plan the BMP administers. The business logic, or logic rules, may be unique to the particular BMP. In some embodiments, the PPIS 300 derives or may be involved with deriving business logic for determining, from the benefits plan data, logic rules unique to each particular BMP that can be used to read from any given benefits plan of the BMP the manner of determining the level of insurance contribution toward a benefit for each of the one or more benefits included in the given benefits plan. The PPIS 300, from the business logic, may further derive a benefits formula based on the manner of determining the level of insurance contribution. In other words, the PPIS 300 may read, extract, or derive the personal benefits plan of the individual in real-time (or near real-time) using the logic rules corresponding to the BMP from which the benefit plan is acquired, and to derive a benefit formula for each benefit included in the personal benefit plan. The PPIS 300 may determine a personal (to the individual) level of coverage of the individual for the given benefit under the personal benefits plan of the BMP or BMPs. The PPIS 300 employs business logic of the particular BMP 327 to determine the particular features of each benefit under the plan. For example, the PPIS 300 may determine how (e.g., formulas) to perform cost-related calculations based on the business logic of the particular BMP and the nomenclature of the individual particular benefits plan. The PPIS 300 may provide, based on the retail price of the inventory data (from the BSP) and the personal level of coverage (from the relevant, personal-to-the-individual benefits plan(s) from one or more BMPs), a balance to be paid by the individual for each inventory item option. See the discussion of FIGS. 10 and 11 for further description of acquiring business logic for a BMP, matching benefits to an inventory of a BSP, acquiring an individual benefits plan, and applying the business logic and inventory to the individual benefits plan to generate correlated benefits plan data.

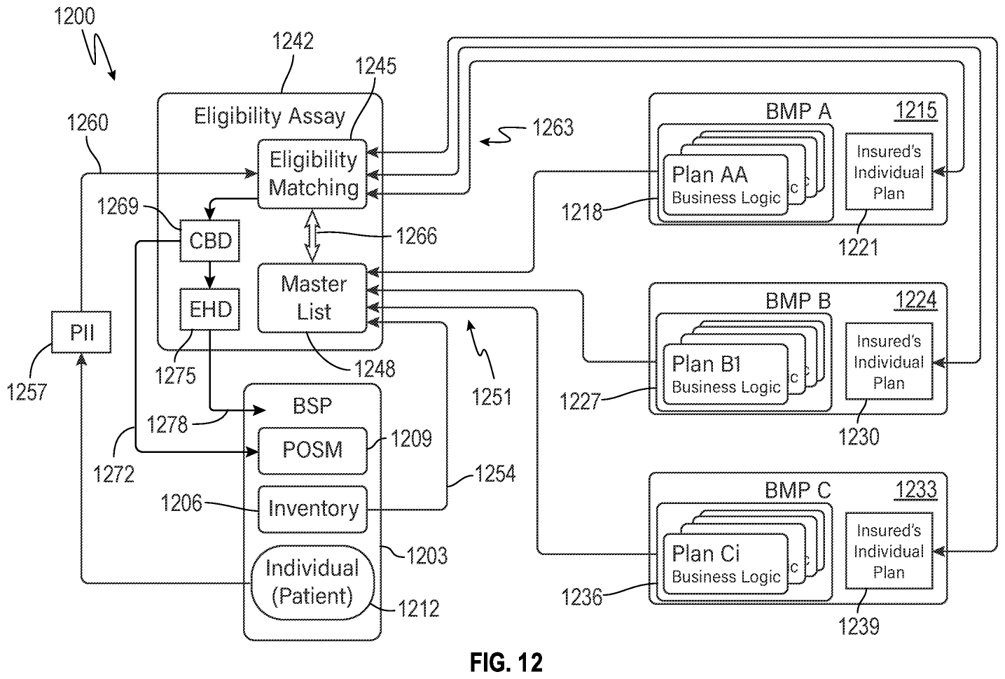

The co-pay calculator tool 350 can receive correlated benefits plan data (“CBD”). The CBD may provide to the co-pay calculator 350 granular and particularized data representing each item of the BSP inventory of items, along with each corresponding benefit of each benefits plan under which the particular individual derives a benefit, and the means to calculate the effect on the cost of each inventory item of the BSP to the particular individual based on the particular benefits plan for that individual and the business logic derived (by the connector tool 325) for the BMP that provides the plan. Generation of the CBD is further discussed in conjunction with FIGS. 11 and 12. The CBD may enable the co-pay calculator tool 350 to display a compilation of services and products of the BSP, each correlated to a particular relevant benefit available to the patient under each benefits plan providing coverage. The co-pay calculator tool 350 may, based on the CBD, render to a display the compilation of correlated services and products, and may provide a means of selecting a particular service or product (or collection of services or products), such as for purchase or to add to an order for potential purchase (e.g. a shopping cart).

The co-pay calculator tool 350 may receive a inventory item option for the individual indicating a given benefit selected for use by or provision to the individual. Furthermore, based on the CBD, the co-pay calculator tool 350 may also display one or more of an insurance contribution amount or rate, allow optional application of a discount amount or rate to a product or order, a co-pay amount or rate, etc. As a current user selects one or more services or products (e.g., by clicking a button or other selection interface tool), the co-pay calculator tool 350 may also provide (e.g., render, communicate) a balance to be paid by the individual 360 for the inventory item option to the BSP and/or the individual, which may then be used in a point-of-sale transaction between the BSP and the individual. The balance to be paid the individual 360 for a inventory item option may comprise co-pay, or a discount from a retail price, etc., or a combination thereof. The balance, for each inventory item of the BSP selected for the individual, may include or otherwise consider one or more of a retail cost, an insurance contribution amount or rate, a deductible, a limit, a discount, a pre-tax amount, a tax amount, a post-tax amount, a co-pay, etc. A balance may be shown, including one or more of the foregoing cost data, for each inventory item of the BSP (and, where relevant, for each BMP), for each selected inventory item of the BSP (and, where relevant, for each BMP), and for a plurality of selected inventory items of the BSP (and, where relevant, for each BMP). The PPIS 300 may render the balance to be paid by the individual for a inventory item option to a graphical user interface (a display).

The BSP (or employee thereof) or patient can apply or remove benefits or insurance contribution at the product-level, to one or more products or services, prior to checkout, based on what is most beneficial to the patient (financially) (product a or product b, where its one or the other) or based on which benefits are most favorable across one or more plans (e.g., based on which plan offers the most benefit, given the order and patient). The PPIS 300 may also be able to recommend the optimal application of policy benefits across one or more policies for a patient.

The PPIS 300 may acquire detailed information about one or more benefits plans provided by each BMP for the particular individual, regardless of the manner in which benefits plans details may be provided (e.g., PDF; XML; HTML; SHTM; unstructured, natural language (human-readable); uniquely structured; etc.) by each BMP. The PPIS may generate, for each benefits plan, a uniform means of displaying the benefits of each plan. Displaying the benefits in a uniform manner, rather than in potentially unique and difficult-to-navigate BMP plan forms may make understanding the benefits easier, and facilitate employing the benefits in a more beneficial way for the individual. When a benefits eligibility search request is received at the PPIS 300, the PPIS 300 may use information personally identifiable (“PII”) to the patient (or an insured or a dependent) to query each of the BMPs. When a patient has eligibility from a BMP, the B SP may acquire from the BMP an electronic version of the particular plan providing coverage for the individual whose PII was provided.

Similarly the BSP inventory for each participating BSP may be acquired, without regard for the manner in which it is stored at the BSP, and placed in or otherwise incorporated into the master list, wherein each inventory item is mapped to a particular benefit (or to a plurality of benefits) of each BMP. Upon identifying a benefits plan for an individual, and deriving the characteristics of each benefit based on the business logic of the relevant BMP, the relevant BSP inventory items and correlated benefits of the benefits plan are made available to a current user of the PPIS 300.

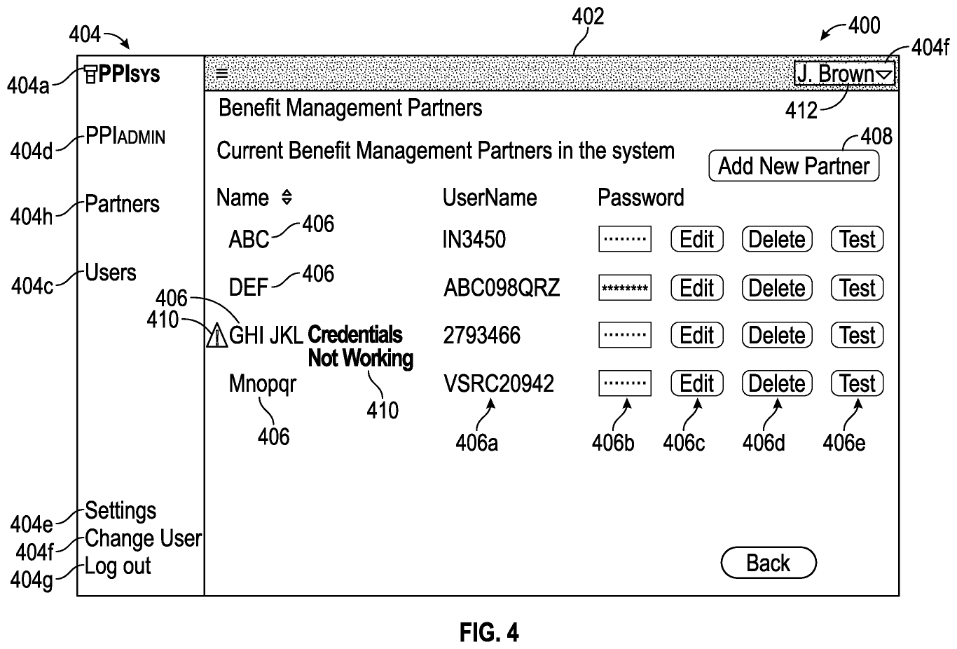

FIG. 4 is an illustration of a BMP administration screen 402 of a BSP (e.g., the BSP 120, 220 of FIGS. 1-3) for a PPIS 400, according to an embodiment of the present disclosure. A menu 404 for navigating through the PPIS 400 is shown. The menu 404 may include a home button 404a, an administration button 404d, a partners button 404h, a users button 404c, a settings button 404e, a change user button 404f, and a logout button 404g. A username 412 for a current user of the PPIS 400 is shown. The current user may be changed by clicking either of the change user buttons 404f shown in the menu 404 or adjacent the username 412. The PPIS administration functions may be accessed by clicking the administration button 404d. The administration screen 402 in FIG. 4 may be accessed by clicking the partners button 404h. A users management screen (not shown) may be accessed by clicking the users button 404c.

The administration screen 402 may be used by an employee or practitioner of a BSP 220 to configure the PPIS 400 to interact with a BMP 418a-418d on behalf of the BSP 120. Four BMPs 418a-418d are shown in FIG. 1; however more or fewer BMPs 418x may be available to a given PPIS 400 or BSP 120. For each BMP 418a-418d, a username 406a and password (placeholder) 406b may be available whereby the BSP 120 may connect with each individual BMP 418a-418d. For each BMP 418a-418d, an associated edit button 406c, delete button 406d, and test button 406e are shown. Clicking on the edit button 406c may, if the current user has appropriate privileges, cause a display to be shown whereby the username 406a and/or password 406b may be edited. Clicking the delete button 406c, by an appropriately privileged current user, may cause the particular BMP 418a-418d to be deleted from the BSP account (a confirmation or cancel opportunity may be presented) in the PPIS 400. Clicking the test button 406d may cause the PPIS 400 to test accessing the particular BMP 418a-418d with the associated username 406b and password 406c.

Clicking the add new partner button 408 may cause a page to be displayed whereby an appropriately privileged current user may enter information about a BMP 418x, including a username 406a and password 406b.

An error condition 410 is shown for BMP 418c indicating the PPIS 400 is unable to connect to the BMP 418c. The error condition 410 comprises an error flag 410a and a configurable error message 410b. The configurable error message 410b in FIG. 4 indicates that the username 406a and password 406b combination is being rejected by the BMP 418c system. The configurable error message 410b may be configured to indicate any of a variety of error conditions. For example, communication errors such as connection rejected by server, connection timeout, no response, no route to server, etc., may cause a particular configurable error message 410b to be displayed, as may some configuration issues. Furthermore, a BMP 418a-418d or the PPIS 400 may be configured to permit a maximum number of queries a particular BPS 220 against a BMP 418a-418d in a predetermined period of time (such as during a subscription period), and reaching the maximum number of queries may generate a particular configurable error message 410b.

When an error such as those mentioned above occurs, in addition to displaying the error flag 410a and a configurable error message 410b, the PPIS 400 may provide notification to an operator of the PPIS whereby troubleshooting may be initiated, and may disable communication with the particular BMP 418a-418d for a period of time or until a resolution is confirmed, etc.

The username 406a and password 406b may hereafter be referred to collectively as credentials 406a, 406b or set of credentials 406a, 406b. Credentials 406a, 406b may be unique to the particular BMP 418a-418b. Furthermore, the BSP 220 may have a single set of credentials 406a, 406b issued by each BMP 418a-418d, and, more particularly, issued by each BMP 418a-418d to a licensed professional practitioner associated with the BSP 220. The users button 404c in the menu 404 may permit an appropriately privileged user 412 to add, edit, or delete users from the PPIS 400. A user is typically an employee, an associate, a manager, a principal, etc., affiliated with the BSP 220. The BSP 220 is able to allow its users access to the PPIS 400 without disclosing to an individual user the principal's credentials 406a, 406b provided by each BMP 418a-418d. The PPIS 400 may be configured to record accesses by each user, that may include logins, logouts, patient benefit queries, claims management, payment system access, copay calculations, etc. Furthermore, a BSP 220 may edit a user's privileges, suspend a user, delete a user, etc., which may improve HIPAA compliance, conformity to business rules, etc. Privilege levels may include two or more tiers. A user assigned a lower privilege tier may be able to perform a limited set of functions in the PPIS 400, while a user assigned an upper privilege tier may permit performing all functions available to the BSP 120 in the PPIS 400. Furthermore, one or more privilege tiers may exist whereby a user may be permitted to perform more functions than any lower privilege tier and less than all functions of any higher privilege tier. By way of example without limitation, at a first tier, a user may be able to access the BMP query functionality of the PPIS 400; at a second tier, a user may be able to perform the lower tier functions and able to edit users of the BSP 120; at a third tier, a user may be able to access all functions of the PPIS 400; etc.

FIG. 5A is an illustration of a search input screen 502 of a PPIS 500, that may be similar in many respects to the PPIS 100, 200, 400 of FIGS. 1-4, according to an embodiment of the present disclosure. A menu 504 is shown, having an eligibility search button 504b, a users button 504c, and a PPIS administration button 504d. The eligibility search button 504b may be displayed on a number of the screens described herein, and other screens. When clicked, the eligibility search button 504b may invoke display of the search input screen 502. The users button 504c may allow the display of users who have been previously entered via the users button 404c of FIG. 4. The users button 504c may allow or otherwise enable convenient switching between users sharing a computer. Clicking the PPIS administration button 504d may invoke the administration screen 402 of FIG. 4.

The search input screen 502 allows a user to enter patient identifying information in order to initiate a benefits information request. A user may manually enter a patient's name 509, typically a last name 509a and first name 509b. The patient's date of birth 510 may be entered in a month field 510a, a date field 510b, and a year field 510c. In some instances, a BMP (see the BMPs 418a-418b in FIG. 4) may require entry of a portion (last 4 digits) of a patient's Social Security Account Number (“S SAN”), for which the SSAN field 511 is provided.

The search input screen 502 further may display information about previously entered searches in a results region 514. In the present example, the last name field 509a has a name entered. Entering information in the name fields 509 causes real time filtering of any results presently available to the current user. Thus, if the current user had recently conducted a search for an individual shown in the name fields 509, the individual would be shown in the results region 514.

Clicking the search button 512 causes the patient identifying information to be captured and sent to the PPIS server (see the PPIS server 140, 240 in FIGS. 1 and 2). Additional data may be captured and sent to the PPIS server, as well, as described above.

Once a search has been initiated, the PPIS 500 may configure the browser to periodically poll the PPIS server 240. Polling of the PPIS server 240 may comprise requesting a status of the request associated with a particular query ID (see the query ID 239b in FIG. 2A). Polling of the PPIS server 240 may permit the browser to be updated in near-real time to display the current status of a search request.

FIG. 5B is an illustration of the search input screen 502 of the PPIS 500 of FIG. 5A, with a search in progress, according to an embodiment of the present disclosure. The results region 514 shows patient identifying information 508 for a current search. In the present example, the browser has polled the PPIS 500 causing partial results to be displayed. More particularly, the BMPs 518a and 518b are shown processing 520; the BMP 518c shows that results have been found indicating the patient may be eligible for some benefits 522 through the BMP 518c; and the BMP 518d is shown having completed with no results found 524, indicating the patient may have no benefit eligibility through BMP 518d. In particular, benefits eligibility may be visually cued in a display rendering, for example, by textual modification (bold, italic, underscore, etc.) and/or colored indicator adjacent to a textual indicator, such as green for a positive eligibility result and red for a negative eligibility result. Such an indicator may generally indicate benefits 522 available to a patient under one or more benefits plans of one or more BMPs 518a-518d.

The PPIS 500 permits the current user to add a search for an additional individual who may be eligible for benefits under the same health-related services plan as the individual shown in the patient identifying information 508. Once the search for the patient has started, the patient information is shown in the results region 514, all BMPs 518a-518d may initially show as processing 520, and an add dependent button (see the add dependent button 525 in FIG. 5C) may be displayed that, when clicked, displays a dependent search field 528. The current user may enter the dependent's name 528a and date of birth 528b, and click the search button 532 to initiate a search for benefits for the dependent.

In FIG. 5B, the results region 514 also shows a recent search result set 534 for a previous search requested by the current user. With no new search being entered as in FIG. 5A, the recent search result 534 wherein the PPIS 500 queried four BMPs 518a-518d. The results from the BMPs 518a-518d indicate benefits 536 available through BMP 518b, and no benefits available 524 through the remaining BMPs 518a, 518c, 518d. Furthermore, relative to the patient identifying information 508, one benefit is shown available 522 through BMP 518c. In particular, each “Service” shown is preceded by an indicator 519a or 519b. The indicator 519a may indicate the associated “Service” is not available, while the indicator 519b may indicate the associated “Service” is available. Similarly, in the search result set 534, the BMP 518b shows benefits eligibility 536 exists for at least the five displayed “Services,” as indicated by an indicator 519c preceding each “Service.” The indicators 519a, 519b, 519c may take any form appropriate to reflect a positive or negative result based on the results returned. For example, and without limitation, a “+” may reflect a positive result while a “−” may reflect a negative result; a particular color (e.g., green) may reflect a positive result while another color (e.g., red) may indicate a negative result; etc. In one embodiment, the indicators 519a, 519b, 519c may be omitted and a text effect (e.g., a text color, etc.) may be used to indicate a positive or negative result.

FIG. 5C is an illustration of the search entry screen 502 of the PPIS 500 of FIGS. 5A, 5B, showing a completed search based on the input of FIGS. 5A, 5B, according to an embodiment of the present disclosure. The menu 504 and results regions 514 are shown. In the results region 514, the search for a patient has completed, as is evidenced by the BMP 518d showing benefits eligibility 536, and the BMPs 518a-518c showing no benefits. Furthermore, the patient whose patient identifying information 508 is shown is also the primary insured 538 for the particular health-related services plan administered by the BMP 518d.

Two dependent beneficiaries 528, 530 are shown in conjunction with the primary insured 538. The current user may have entered a search request for, for example, the first dependent 528 by clicking the add dependent button 525 and entering dependent identifying information in a dependent search field (see the dependent search field 528 in FIG. 5B). Alternatively, as the PPIS 500 was querying, via the virtual servers and ephemeral containers (see the virtual servers 241, 261, 275 and ephemeral containers 243, 263, 277 in FIG. 2A), the BMP 518d, the virtual server 241 determined, as described in FIG. 2A, a dependent of the primary insured 538 exists within the BMP 518d and the PPIS 500 automatically executed the additional logic described above with reference to FIG. 2A to acquire dependent identifying information and benefits eligibility 536. This functionality is particularly useful when the BSP 220 may be provisioning service(s)/product(s) to multiple members of a family under the same health-related services plan. For example, if the primary beneficiary 538 and both dependents 528, 530 need provisioning from the same BSP 220 at or about the same time, a single search query may produce benefits eligibility for all three persons 538, 528, 530 in a few moments, with a high degree of accuracy, confidence, and repeatability.

Below the current search result set 538, 528, 530, a first recent search result set 534 is shown, as is a second recent search result set 540. The second recent search result 540 shows a refresh eligibility button 542. The refresh eligibility button 542 may be displayed, for example, when a designated period of time has elapsed since the relevant search request was sent to the PPIS 500. Clicking the refresh eligibility button 542 may cause the PPIS 500 to perform the benefits search anew so that current benefit eligibility may be acquired. This feature may be particularly useful when the BSP 220 has a recurring or returning patient.

FIG. 5D is a search entry screen 502 with a benefits eligibility overview screen 544 for the PPIS 500 of FIGS. 5A-5C, according to an embodiment of the present disclosure. The menu 504 is shown for reference. Patient identifying information 508 is displayed on the benefits eligibility overview screen 544, as is the BMP 518d. A benefits eligibility overview screen 544 may be available for a primary insured and related dependents (see the primary insured and dependents 538, 528, 530 in FIG. 5C). In one embodiment, if a patient is eligible for benefits under multiple plans identified by the PPIS 500, the benefits eligibility overview screen 544 may consolidate and display together the information from the plurality of plans. The benefits eligibility overview screen 544 may display one or more benefit classes 546, and benefit types 548 available under each benefit class 546, and each particular benefit (procedure, service, appliance, etc.) 550 available under each benefit type 548. The PPIS 500 may be configured to display benefit classes 546, benefit types 548 and benefits 550 available to the patient under the particular benefits plan. A summary statement 552 may be shown for each particular benefit 550. A view plan snapshot button 554 may be clicked to access further information about benefits 550 under the plan. A copay calculator button 556 may be clicked to access the copay calculator described below.

FIG. 5E is an illustration of a plan snapshot screen 558 of the PPIS 500 of FIGS. 5A-5D, according to an embodiment of the present disclosure. The plan snapshot screen 558 may be invoked by clicking the view plan snapshot button 554 of FIG. 5D. The menu 504 is shown for reference. Patient identifying information 508 and the BMP 518d are displayed. The plan snapshot screen 558 shown in FIG. 5E comprises a patient eligibility tab 560 and a plan benefits tab 562. The patient eligibility tab 560 is currently active. BSP information 564 is shown. For a BSP 518a-518d having multiple practitioners or providers, a menu 566 allows selection of a particular practitioner or provider. A date of service 568 may be entered. A benefit type 546 and/or benefit class 548 may be shown as selectable tabs whereby benefits 550 related to the particular benefit type 546 or benefit class 548 may be displayed. For each benefit 550 displayed, a flag 572 indicating whether or not a particular benefit is currently available for the patient. An eligibility date 574 is shown for each benefit 550. If a particular benefit 550 is currently not available to a patient, the eligibility date 574 may indicate when the patient becomes eligible for the particular benefit 550. A benefit (or provisioning) frequency 576 for each benefit is also shown. Each benefit 550 has an associated selection box 570. Clicking a selection box 570 denotes inclusion of the associated benefit to be provided to the patient, used for preparing a claim against the BMP 518d, and for computing patient copays. A submit claim button 578 and a copay calculator button 556 may be included. The submit claim button 578 may trigger automatic preparation of a claim for presentation to the BMP 518d. Clicking the copay calculator button 556 serves to invoke the copay calculator, described below.

FIG. 5F is an illustration of the plan snapshot screen 558 of FIG. 5E, with the plan benefits tab 562 selected, according to an embodiment of the present disclosure. The menu 504 and the patient eligibility tab 560 are shown for reference. The patient identifying information 508 and the BMP 518d remain displayed with the plan benefits tab 562 active. The plan benefits tab 562 may list benefits 550, in-network details 582, and out-of network details 584. The in-network details 582 and out-of-network details 584 may comprise information such as, e.g., copay amounts, limits, etc. The copay calculator button 556 remains available on the plan benefits tab 562.

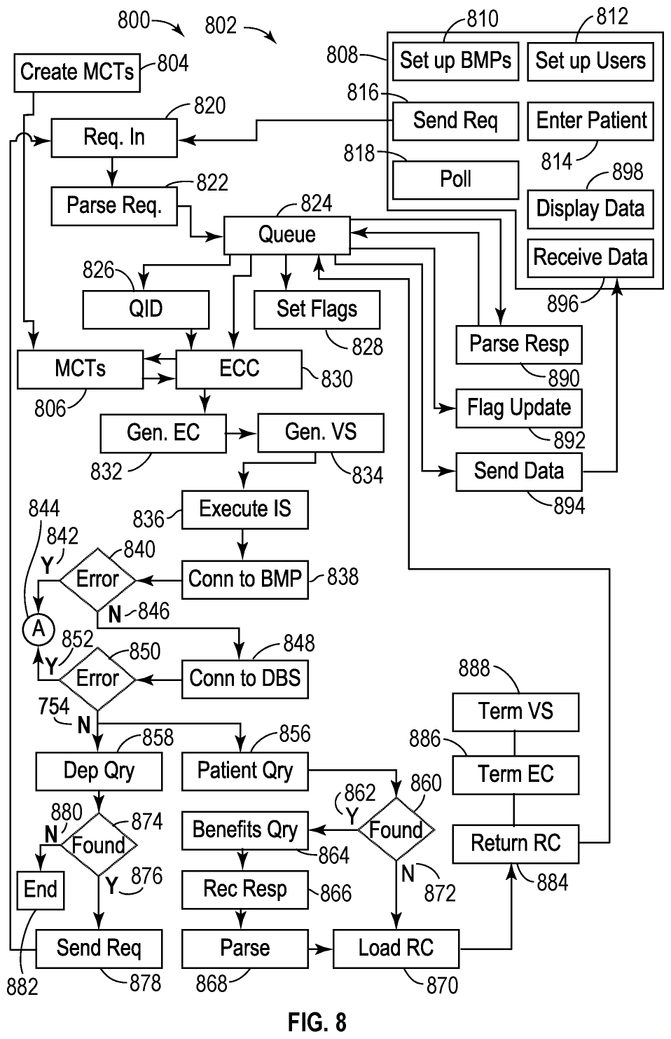

The benefit classes, benefit types, benefits, eligibility status, eligibility dates, benefit/provisioning frequency, in-network and out-of-network details of FIGS. 5A-5F, and other related information, may be obtained by the PPIS 500 by querying the BMPs 518a-518d as described in discussion FIGS. 1 and 2, and in the discussion of FIGS. 8, 11 and 12, below.

FIG. 6A is an illustration of a copay calculator screen 602 of a PPIS 600 that may, in many respects, be similar to the PPIS 500 of FIGS. 5A-5F, according to an embodiment of the present disclosure. The copay calculator screen 602 includes a menu 504, patient identifying information 608, and indication of the BMP 618d. Based on the information retrieved by the PPIS 600 from the BMPs 518a-518c of FIG. 5A and 618d, the copay calculator compiles the benefit classes (see the benefit class 546 of FIGS. 5D, 5E), the benefit types 648, and benefit details. The benefit details may comprise a benefit, an associated copay, limit(s), restriction(s), requirement(s), frequency, etc. (see the benefit 550 in FIGS. 5D-5F, the benefit information 552 in FIG. 5D, the eligibility flag 572, eligibility date 574, and benefit/provisioning frequency 576 in FIG. 5E, and in-network and out-of-network details 582, 584 in FIG. 5F). The copay calculator 602 may also be configured (described elsewhere herein) with fees and prices for provisioning by the BSP (see the BSP information 564 in FIG. 5E). The copay calculator 602 associates the BSP fees and prices to the relevant benefits information and computes costs and generate the copay calculator screen 602.

The copay calculator 602 shows a plurality of benefit classes 648 and associated benefits 650. For each benefit 650, the copay calculator 602 may display the BSP fee 652 associated with the particular benefit 650. The copay calculator 602 may also display the patient's copay 654 for the particular benefit 650. The copay calculator 602 may also display a select/deselect toggle 656, 658 for each benefit. The select/deselect toggle 656/658 indicates the state that will be invoked when the toggle 656/658 is clicked. In other words, when a benefit 650 has been selected, the toggle 656/658 will be the deselect toggle 658, and clicking the deselect toggle 658 will both deselect the particular benefit 650 and cause the select toggle 656 to be displayed. A first state of the toggle 656/658 and of each benefit 650 may be set by checking the relevant check box(es) patient eligibility screen (see the check boxes 570 and patient eligibility screen 560 in FIG. 5E). A review/checkout button 660 may be included that, when clicked, may display the copay calculator screen 602 in FIG. 6B.

FIG. 6B is an illustration of a copay calculator review/checkout screen 602 of the PPIS 600 of FIG. 6A, according to an embodiment of the present disclosure. The menu 604 is shown for reference. The patient identifying information 608 and an indicator of the BMP 618d for the current transaction are shown. The review/checkout screen 602 may display a plurality of benefit types 646, along with benefit classes 648 having benefits (see the benefits 650 in FIG. 6A) selected in the copay calculator screen 602 of FIG. 6A. For each chosen benefit class 648, a total for BSP fees 662, a total for patient copays 664, a total for taxes 666, and a total pay amount due from the patient 668 is shown. The PPIS 600 may be configured to acquire a local tax table for the BSP (see the BSP information 564 in FIG. 5E) and may further identify which benefits are taxable under the relevant tax scheme. A revisit button 670 is also associated with each benefit class 648 that, when clicked, may return the browser to a previous copay calculator screen, such as the copay calculator screen 602 of FIG. 6A, to a benefit information and selection page, such as the plan snapshot screens 502 of FIGS. 5E and 5F. A benefit type subtotal 672 for each benefit type is also shown for each benefit type 646. The review/checkout screen 602 may also include a summary overview 640 showing the total BSP fees 674, the total copay amount 676, the total taxes 678, and total payable amount 680 due for the current provisioning is shown. The summary overview 640 may also include a total savings 682 for the current provisioning based on the health-related services plan information provided by the BMP 618d, the BSP fee data, and local tax information.

The review/checkout screen 602 may also have a cancel button 684 and a payment and claim button 686. The cancel button 684, when clicked, may cancel all benefit selections made under the information provided by the BMP 618d. In one embodiment, clicking cancel button 684 may also allow the current benefit selections to be stored for future use. In one embodiment, the review/checkout screen 602 have a save button (not shown) to store the current benefit selections for future use. The payment and claim button 686 may be clicked to initiate preparation of a claim to be submitted to the BMP 618d based on the benefit selections for the current transaction, and present a payment opportunity to receive payment from the patient (or to place the transaction in a billing status for future payment).

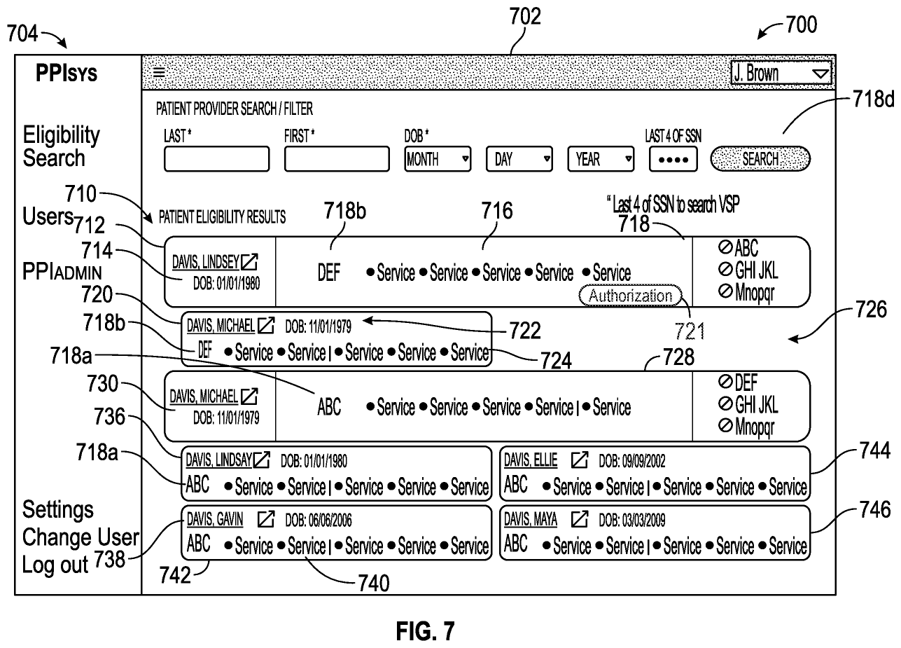

FIG. 7 is an illustration of a search entry screen 702 of a PPIS 700 similar to the PPIS 500, 600 of FIGS. 5A-6B and showing benefit information from more than one of the plurality of BMPs 718a-718d, according to an embodiment of the present disclosure. Benefit information is shown for two health-related services plans 710, 726, with the first plan 710 administered by the BMP 718b, and the second health plan 726 administered by the BMP 718a. The first plan 710 is shown with a primary insured block 712 comprising patient identifying information 714, and a dependent block 720 comprising dependent identifying information 722. The second plan is shown with a primary insured block 728 comprising patient identifying information 730 and four dependent blocks 736, 742, 744, 746. Each of the dependent blocks 736, 742, 744, 746 is associated with a different dependent. Dependent identifying information 738 is labeled for the dependent block 736.

A search request may have originally been implemented to obtain benefit information for the individual shown in the primary insured block 712 of the first plan 710. The PPIS 700, as previously described in conjunction with FIGS. 2A and 2B, may have identified this individual as a primary insured at BMP 718b and as a dependent insured at BMP 718a. Furthermore, the PPIS 700 may have identified that other beneficiaries are included in the same health-related services plans of BMPs 718a, 718b as the patient for whom the search was initiated. The PPIS 700 executed additional search functionality to locate information regarding the dependent(s) covered by the same health-related services plan. In other words, once the PPIS 700 learned of the existence of a dependent or primary insured other than the individual for which the search was initiated, the PPIS 700 extended its search to identify each dependent and primary insured, and to retrieve benefit information for each of these by querying each of the BMPs 718a-718d.

In the present example, the BMP 718b administers the first plan 710 shown. The primary insured block 710 identifies the primary insured in the plan 710 administered by the BMP 718b, and provides a benefits list 716 for the primary insured 714. The dependent block 720 of the first plan 710 similarly shows a dependent 722, and a benefits list 724. The dependent 722 is identified as the primary insured 730 in the primary insured block 728 of the second plan 728. The second plan 728 is administered by the BMP 718a. Four dependents are shown in dependent blocks 736, 742, 744, and 746. Each dependent block 736, 742, 744, 746 provides a dependent name 738 and a benefits list 740. Each primary insured block 712, 728 and dependent block 720, 736, 740, 742, 746 also indicates the administering BMP, either 718a or 718b, and also which BMPs of the plurality of BMPs 718a-718d that do not provide benefits under a plan for the respective primary insured 714, 730. In the illustrated example, the primary insured 714 of the first plan 710 is a dependent (in the dependent block 736) in the second plan 726. Similarly, the dependent 722 shown in the dependent block 720 of the first plan 710 is the primary insured 730 in the second plan 726.

In the present example, the BMP 718b requires a service provider, such as a BSP, to obtain authorization prior to providing a service under a benefit plan administered by the BMP 718b. The PPIS 700 adds an authorization button 721 to the display. The current user of the PPIS 700 may click (or otherwise activate) the authorization button 721 to initiate an automated authorization request whereby authorization may be obtained to provide covered services under the relevant benefits plan. The PPIS 700 also allows managing authorizations, which can include pulling authorizations and/or removing authorizations for a group (e.g., a family) or for individuals, including individuals in a group. An authorization button may be provided for each individual.

FIG. 8 is a method diagram 802 for a PPIS 800 similar to the PPIS 100-700 of FIGS. 1-7, according to an embodiment of the present disclosure. One or more master container templates (“MCTs”) are created 804 and stored 806 at a repository. A BSP is configured 808. In configuring 808 the BSP, one or more sets of BMP credentials may be entered 810 whereby benefit searches at BMPs may be authorized. One or more users may be added 812. The users may be permitted to initiate benefit searches against BMPs without the BMP credentials being exposed to the users. A user enters 814 patient identifying information into a search entry screen and sends 816 a search request. Once the search request is sent 816, the BSP system may begin periodic polling 818 to ascertain the current status of the search request. The request is received 820 by a PPIS server, and is parsed 822. The parsed request is passed to a queue 824. A unique query token (“QID”) may be generated 826. In one embodiment, to send 816 the search request, the BSP system connects with a PPIS server and in a manner (e.g., using a particular port, protocol, etc.) that notifies the PPIS server that the BSP system is preparing to send 816 the search request. The PPIS server may cause the queue 824 to generate the unique query token, and the unique query token may be returned to the BSP system for insertion into the search request, after which the search request may be sent 816. In one embodiment, after the search request is received 820, the unique query token may be generated 826 and returned to the BSP system. In one embodiment, the BSP system may generate a BSP query token and include the BSP query token in the search request. When the search request is received 820, the unique query token may be generated 826 and may be associated with the BSP query token either by the PPIS server, the BSP system, or both.

The queue 824 may generate 828 a flag set associated with the unique query token. The flag set may comprise a plurality of indicators, each related to a particular status for the search query associated with the unique query token. By way of example without limitation, a flag may be generated related to each BMP, and may be initially set to reflect a default status, e.g., pending. The initial pending flag may cause a response to polling 818 to indicate the current search query is pending, which, in turn, may permit display of a processing state at the BSP system (see processing 520 in FIG. 5B).

The ephemeral container controller may be activated 830. The ephemeral container controller may request and receive 806 a copy of each MCT. The ephemeral container controller may generate and configure 832 an ephemeral container for each BSP. The ephemeral container controller may generate a plurality of ephemeral containers simultaneously, or nearly so. Each ephemeral container is agnostic or unaware of the existence, state of operation, etc., of every other ephemeral container. Each ephemeral container may operate or function in a wholly independent of any other ephemeral container, the results of any other ephemeral container, etc. Configuration of an ephemeral container comprises inserting into the ephemeral container from its parent MCT machine-executable instructions (“logic”) for the particular BMP, patient identifying information from the search query, the unique query token, and results shell or results container. The logic may comprise server operating system-level instructions (e.g., connection and communication protocols) and database system instructions (e.g., query language) particularly configured for the BMP server with the ephemeral container is to be associated.

A virtual server instance is spawned 834 for each BMP server, and the particular ephemeral container is added to the virtual server. Each virtual server may be configured to parse the ephemeral container and to execute 836 the logic, including utilizing the patient identifying information. In one embodiment, the logic may comprise literal machine-executable instructions to be passed by the virtual server from the ephemeral container to the BMP server. In one embodiment, the virtual server may comprise a library of machine-executable instructions that may be individually invoked from a logic segment of the ephemeral container, whereby the a logic segment may be correlated to and invoke a particular machine-executable instruction from the library of the virtual server and the virtual server passes the machine-executable instruction to the BMP server.

The virtual server attempts to connect 838 to the BMP server. If an error arises 840, the virtual server may attempt to resolve the error based on industry connection/communication standards (e.g., attempt again after a pre-defined period of time for a predetermined number of attempts) before setting an error 842 and passing the error to an error handling method A 844. If the virtual server successfully connects 846 to the BMP server, the virtual server may then attempt to connect 848 to a database system of the BMP server. If an error arises 850 that the virtual server cannot resolve 852, the error may be passed to the error handling method A 844. If the virtual server successfully connects 854 to the BMP server database system, one or more database queries may be passed 856 to the database system to determine whether 860 the patient identifying information is associated with a benefits plan. If the patient identifying information is not 872 associated with a benefits plan of the BMP server, the virtual server may load 870 the results container with an indicator that the patient could not be found in a benefits plan. If the patient is found 862 in a benefits plan, the virtual server may pass 864 one or more queries to the database system to acquire benefits plan data, which may include data defining the scope, limits, etc., of benefits. The benefits plan data may be received 866 from the database system of the BMP server and parsed 868 by the virtual server utilizing logic from the ephemeral container. The logic from the ephemeral container may facilitate parsing the benefits plan data to enable the virtual server to load 870 the results container with benefits plan data in a format useable by the PPIS regardless of the format in which the benefits plan data may be acquired and/or stored on the BMP server database system.

One or more queries may be sent 858 to the database to ascertain 874 if any other individual—dependent or primary insured—is enrolled in the same benefits plan as the patient indicated by the patient identifying information. If no other individual is found 880 for the same benefits plan as the identified patient, the dependent query ends 882. If at least one other individual is found 876, the logic of the ephemeral container may cause or enable the virtual server to provide a subsidiary request to the PPIS server to initiate, in essence, a recursive search. The virtual server may also include a dependent-found flag when the results container is loaded 870. The subsidiary request carries the unique query token, functioning as a connecting key to relate the subsidiary request to the initial request, and at least some identifying information of the other individual. The subsidiary request, when received 820 by the PPIS, may be handled in much the same way as the initial request except that the unique query token may be used to associate the individual identified in the subsidiary request with the patient of the initial request. Furthermore, the logic within the ephemeral container may comprise one or more queries directed toward identifying the relationship of the patient (whether in the initial request or the subsidiary request) to the benefits plan. In other words, to determine whether the individual named is a primary insured or a dependent-insured.

Once the results container is loaded 870, the virtual server returns 884 the results container to the PPIS. The virtual server may await a confirmation from the PPIS that the results container has been successfully received. Once the virtual server receives this confirmation, the virtual server may signal the ephemeral container whereby the ephemeral container is able to self-destruct. The virtual server may likewise self-destruct and return or otherwise free computing resources for the PPIS.

The queue may parse, or cause to be parsed, 890 the data within the results container. From the parsed data, the queue may identify that the patient is not a beneficiary of a benefits plan administered by the BMP corresponding to the particular virtual server, ephemeral container, and results container; or that the patient is a beneficiary of a benefits plan of the particular BMP and that the patient is a primary insured or dependent-insured. The parsed data may include a dependent-found flag indicating that a recursive search is in progress. Because both the initial request and the subsidiary (recursive) request are handled by disparate virtual servers, it is possible, due to communication or other latencies, that the recursive request completes before the initial request. Sharing of the unique query token, whether appearing in the results container as the unique query token or as a connecting key, may enable or otherwise permit the queue to update or set one or more flags to effect answers to polling 818. In other words, the queue may change a status indicator to complete, meaning the search request—whether as an initial search or a recursive search—has completed. The queue may set a flag indicating a dependent was found. Note that, at the queue, a dependent-found state may be expressed within the parsed data of a results container identifying the named patient of the particular data is a dependent within the particular plan, or that the named patient is primary insured and a dependent was found. Setting of a dependent-found flag may permit updating a browser display to show one or more dependent blocks in the results region of the search inquiry screen (see the dependent blocks 528, 530, results region 514, and search inquiry screen 502 in FIG. 5C. The queue may, based upon receiving a results container indicating the patient was not found, set a not-found flag.

Polling 818 of the PPIS may note any addition of a flag or change of a flag, and may result in sending 894 data to the BSP browser corresponding to any new flag or changed flag. When the data is received 896 at the BSP the display may be updated 898 to reflect the new information. For example, setting of a dependent-found flag may cause data 894 to be sent to the BSP browser whereby the BSP browser adds a dependent block 528, 530. Changing a flag from “processing” to “not found” may cause data to be sent 894 to and received 896 at the BSP browser whereby a “processing” icon may be deleted from the display and a “not found” icon may be added. In another embodiment, the “processing” flag may change to a “complete” flag (as in a binary state change) in conjunction with another flag indicating “not found” or “found,” essentially producing corresponding browser changes (see FIGS. 5B, 5C).

Because the PPIS employs as many virtual servers as there are BMPs, with each virtual server corresponding to one BMP, and each virtual server operates agnostically with reference to the existence of the other virtual servers, the PPIS is able to identify an instance wherein a patient may be an insured in multiple benefits plans administered by different BMPs. When such is the case, the browser at the BSP may be updated to resemble the screen in FIG. 7.

Once at least one search has completed and returned benefits plan data for a benefits plan covering the patient (whether the patient in an initial search or in a recursive search), benefits information may be sent 894 to and received 896 by the BSP, whereby the copay calculator may be enabled. See the discussion of FIGS. 3, 5D-6B and 10 regarding the copay calculator.

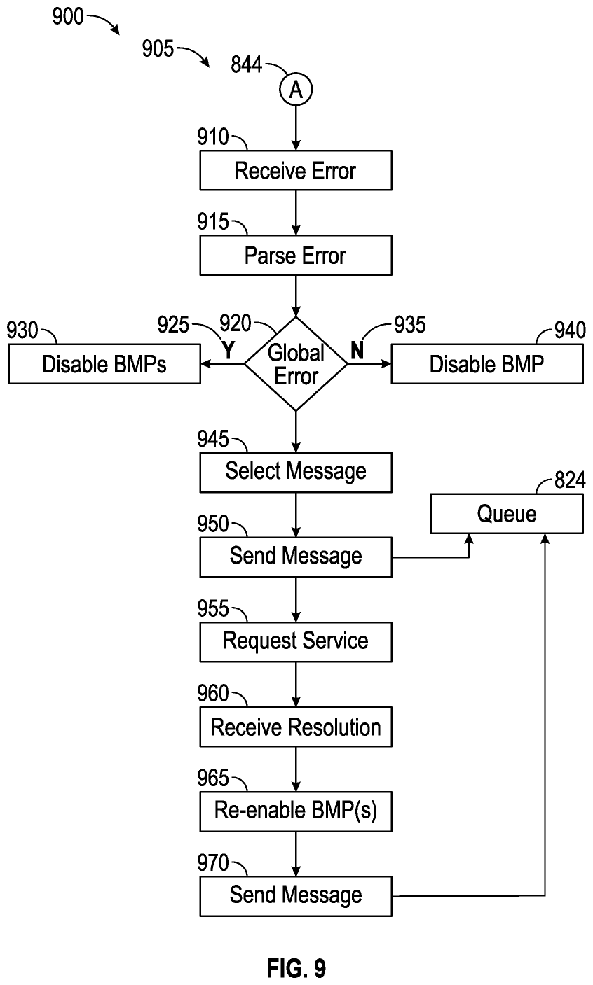

FIG. 9 is an error handling method diagram 905 for a PPIS 900 that is similar in many ways to the PPIS 100-800 of FIGS. 1-7, according to an embodiment of the present disclosure. Error handling may be invoked on a signal, such as receipt or non-receipt of a particular message during execution of an instruction set of an ephemeral container (see execution of the instruction set 836, error 840, 850 in FIG. 8). By way of non-limiting examples, errors may arise when a communication path cannot be established (no route to host), credentials for logging in to a BMP are invalid, insufficient or incorrect information has been provided as input to a BMP, a BMP server (or web portal) does not respond within an allotted period (timeout), etc. A connector 844 in FIG. 8 is also shown in FIG. 9 to relate the error handling method 905 of FIG. 9 to the method diagram 802 of the PPIS 800 of FIG. 8. Stated otherwise, the error handling method diagram 905 represents a child method of the method diagram 802 of FIG. 8. Error handling generally commences with receipt 910 of an error flag. An error flag may occur upon receipt of a particular message from a system or subsystem of the PPIS 900, or from a system or subsystem in communication with the PPIS 900. An error may also occur upon non-receipt of a message, or upon receipt of incomplete or corrupted message. In an instance wherein non-receipt of a message, or receipt of corrupted or incomplete message gives rise to the error condition, a subsystem (e.g., an ephemeral container, such as an ephemeral container 243, 263, 277 of FIG. 2A) generates an error message.

The error message may be parsed 915 to identify one or more characteristics of the error. If the error message is related to one BMP or a plurality of BMPs (see the BMPs 252, 254, 256 of FIG. 2A) comprising less than the complete set of BMPs queryable by the PPIS 900, a “disabled” flag may be set for each affected BMP. If the error message is general to all BMPs queryable by the BMP, a “disabled” flag may be set for all BMPs individually, or a collective “disabled” flag may be set. A message may be selected 945 and sent 950 to the queue (see the queue 824 in FIG. 8). The message may convey information to the queue 824 regarding the nature of the error encountered and identifying each BMP flagged as “disabled.” For example, if a connection attempt is rejected due to a credentials mismatch, a message may be generated reflecting this status, which may result in flagging of the particular BMP as “disabled” with regard to the BSP whose credentials are currently invalid, and may further generate a notification for the BSP to inform the BSP that the credentials are currently invalid. Flagging a BMP as “disabled” may prevent the ephemeral container controller 233 from generating ephemeral containers 243, 263, 277 for the “disabled” BMP. In one embodiment, an ephemeral container 243, 263, 277 which traps and/or transmits an error message may self-destruct upon receipt of an ACK (acknowledgment) from the PPIS 900. In one embodiment, the ephemeral container controller 233, or another process of the PPIS 900 may send a “destruct” signal to the ephemeral container 243, 263, 277 that sent the error message. Furthermore, when a BMP is flagged “disabled,” the ephemeral container controller 233, or another process, may be configured to terminate any ephemeral container 243, 263, 277 for the particular BMP once a particular period of time elapses and the particular ephemeral container(s) 243, 263, 277 have not otherwise responded. The PPIS 900 may request 955 human intervention directed toward rectifying the cause of the error. In other words, one or more computer engineers, technicians, troubleshooters, etc., may be notified of the occurrence of the error to facilitate addressing any condition internal to the PPIS 900 that may have caused the error, any communication error between the PPIS 900 and the particular BMP(s), or to interact with the BMP to notify the BMP of an apparent error condition at the BMP. Intervention is intended to resolve any condition producing the error, as well as any cascade error(s) that may arise from the initial error. Once the error(s) has/have been resolved, a notification is provided to the PPIS 900 that, when received 960 may cause or permit removal of a “disabled” flag (or setting an “enabled” flag) for each affected BMP. With the “disabled” flag removed (or the “enabled” flag set), a message may sent 970 to the queue 874 whereby the queue becomes configured to operate nominally with respect to the particular BMP (cause the ephemeral container controller 233 to resume generation of ephemeral containers 243, 263, 277 for the now enabled BMP). Furthermore, the queue 824 may be configured to permit storing or retaining information about queries that were initiated against the disabled BMP, or which did not return a result from the disabled BMP. Upon removing the “disabled” flag or setting the “enabled” flag, the queue may cause re-processing of the relevant queries for the particular BMP.

FIG. 10 is a copay calculator method diagram 1004 for a PPIS 1000 that is similar in many ways to the PPIS 100-900 of FIGS. 1-9. Benefit data is acquired 1008 by the PPIS 1000 from at least one BMP (see the BMPs 252, 254, 256 of FIG. 2A). Inventory data for the BSP (see the BSP 220 of FIG. 2A is acquired 1012 by the PPIS 1000. Inventory data may comprise any provisioning that the particular BSP is able to offer, possibly including provisioning for which a particular health-related services plan may not offer a benefit. Tax data may be acquired 1016 by the PPIS 1000 in any instance in which tax may be collectible. The benefit data, inventory data, and tax data may be integrated 918 by the PPIS by comparing the benefit data from each benefits plan under which the patient may be eligible to receive benefits to the provisioning which the BSP 220 is able to offer. The integration 918 may further apply tax to any provisioning taxable under a relevant taxing authority.

A benefits plan may provide one of more benefit types. A benefit type may be an overarching collection of related benefit classes. A benefit class may represent a collection of related benefits. From the integrated data, the PPIS 1000 may identify 1020 each available benefit type of a benefit plan under which the patient may be eligible for benefits. The PPIS 1000 may further identify 1024 each benefit class within each benefit type. The PPIS 1000 may identify 1028 each benefit within each benefit class for which the patient may be eligible. The PPIS 1000 may correlate 1032 any BSP-available provisioning to patient-eligible benefits for each benefit within each benefit class and benefit type. BSP-available provisioning may be rendered 1036 to a display screen, along with benefit details, if any, which relate to each BSP-available provisioning. In other words, the integration of data from the BSP, the BMP, and, if applicable, tax data may permit correlation of these data to permit rendering 1036 to a display each provisioning offered by a BSP along with the BSP's fee, any benefit related to each provisioning (e.g., a copay amount or percentage, a discount rate, a maximum benefit amount, etc.), and a patient cost for the provisioning in view of the BSP's fee, the benefit, and taxes (where applicable). The copay calculator may also render to the display a means of obtaining further details about the benefit, a means of selecting or deselecting 1040 a benefit to use, etc.

For each selected benefit, the copay calculator may automatically apply 1044 the BSP's fee, may apply 1048 any benefit limit, may apply 1052 a discount (e.g., a benefit-driven discount, a BSP-offered discount, etc.), may apply 1056 a copay amount for the provisioning, and may determine 1060 whether the particular provisioning is taxable and, if the provisioning is taxable 1064, apply 1068 the correct tax amount.

The copay calculator, by integrating and correlating benefits plan data, BSP inventory, and tax data to permit particular selection of benefits for the patient to use, affords a number of particular improvements above current methodologies. For example, benefits from benefits plans of multiple providers can be viewed together to enable an informed decision as to selection of benefits to use for a particular provisioning. Furthermore, patient-borne costs are automatically calculated and displayed, eliminating errors from improper keying of calculations, improper tax application, etc. and enabling the patient to shop or browse the inventory of services and products with respect to their copay pricing, as opposed to shopping with no pricing or retail pricing. This is done by pre-calculating the copay amounts for a given patient for their associated BMP benefits plan(s) across an entire BSP inventory of services, supplies and products (potentially in real-time, or immediately) upon or after the PPIS performing the initial patient BMP eligibility search. Services may be authorized (for provision by the provider BSP, related to a subsequent claim) by the PPIS at the BMP, upon the BSP's request via the PPIS interface, and claims may be prepared and delivered to a BMP automatically at the time of provisioning and the benefits used, speeding payment to the BSP and timely updating of benefit use at the BMP. In one embodiment, the patient, using a personal computing device, a web-enable mobile phone or tablet, may be able to use the PPIS to compare BSP offers. In other words, the patient may institute an eligibility search and select to view BSP inventory corresponding to (a) particular benefit(s) of any benefits plan offering a related benefit. In this way, in addition to making optimal use of benefits available under one or more plans, the patient may be able to select a BSP offering a better selection of BSP inventory, or preferred pricing (after application of benefit(s)), a combination of BSP inventory and pricing based upon the patient's particular preference, etc.

The copay calculator, based on the foregoing integration, correlation, and selection of benefits may calculate, automatically, and display 1072 the BSP's fee, benefit amount, and patient copay for each benefit. The copay calculator may also calculate and display 1076 subtotal amounts for all selected benefits within each particular benefits class, and may calculate and display 1080 subtotals for each benefit type. The copay calculator may calculate and display 1084 a total for all BSP fees, all benefit amounts applied to the service(s), all copay amounts (including tax for any taxable amounts under the relevant taxing authority), and may further display a total amount saved by the patient by virtue of the benefits applied and the provisioning selected.

FIG. 11 is a relational diagram of a PPIS 1100, according to one embodiment. The PPIS 1100 may be similar in at least some respects to one or more of the PPISs 100, 200, et seq. described above in reference to FIGS. 1-10. FIG. 11 illustrates creation (and maintenance) of a master list 1172 and a logic library 1180. The master list 1172 (e.g. a super connector master list) may provide a mapping between inventory items 1174 of BSPs and benefits offered under plans of BMPs, as will be explained more fully. The logic library 1180 may comprise business logic or logic rules 1183 that can be used to read or otherwise process a benefits plan to extract current (up-to-data) benefits plan information for a patient. The logic library 1180 is shown as a separate component in FIG. 11, and in other embodiments of a PPIS the logic library may be integrated with the master list 1172. The PPIS 1100 of FIG. 1100 includes a plurality of BMPs 1110, 1120, 1130, 1140 from which benefits plan data is received, one or more BSPs 1150 from which inventory data is received, and a connector tool 1160 to create a master list 1172 and/or a logic library 1180 by mapping inventory data to benefits plan data.

A plurality of BMPs, including BMP 1 1110, BMP 2 1120, BMP 3 1130, and BMP 4 1140, are shown in FIG. 11 as providing or otherwise making accessible benefits plan data 1111, 1121, 1131, 1141. The BMP 1 1110 may employ a computer server (as discussed elsewhere herein) to store benefits plan data 1111, which may include or from which may be derived categorization 1112 and business logic 1114, among other data. The categorization 1112 may represent or inform of categories of services, products, etc., collectively, benefit items, constituting benefits under one or more benefits plans of the BMP 1 1110. The categorization 1112, according to some embodiments, may include categories of products or services. For example, in vision care the benefits may be categorized or otherwise organized as exams, frames, lenses, contacts, and the categorization 1112 may be included or otherwise inherent in the benefits plan data 1111. The categorization 1112, according to some embodiments, may include families, groupings, or other sub-categories of types of products or services. For example, in vision care, categorization may include singe vision lenses, bifocal lenses, and trifocal lenses. In some embodiments, categorization 1112 may indicate that a single benefit item of a BMP maps to a single inventory item 1174 in the master list 1172 and a different single benefit item of a BMP maps to a plurality of inventory items 1174 in the master list 1172. The categorization 1112 of the benefits plan data 1111 can flexibly accommodate and communicate these varied forms of mappings to allow mapping inventory items 1154 across various insurance platforms.

Business logic 1114 comprises logic whereby a benefit for an individual under a benefits plan may be determined, along with features of the benefit (e.g., specifications of a service or product, a deductible amount, an insurance contribution amount or rate, a copay amount or rate, etc.). In some embodiments, the business logic 1114 may not be separately collected or stored by the BMP1 1110, but rather may be included in or inherently in benefits plan data 1111 of the BMP 1 1110. In some embodiments, the business logic 1114 includes one or more rules, guidelines, algorithms, methods, processes, forms, templates, and the like.

The BMP 2 1120 may similarly employ a computer server (as discussed elsewhere herein) to store benefits plan data 1121, which may include or from which may be derived categorization 1122 and business logic 1124, among other data for the benefits plans the BMP 2 1120 administers. Likewise, the BMP 3 1130 and BMP 4 1140 also may include a computer server to store benefits plan data 1131, 1141 respectively, which may include categorization 1132, 1142, respectively, and business logic 1134, 1144, respectively. FIG. 11 presents 4 BMPs 1110, 1120, 1130, 1140, and this is for convenience of the disclosure and not by way of limitation. The PPIS 1100 may similarly function with fewer or more BMPs.

The BSP 1150 (which may be representative of one or more BSPs) controls, holds, and/or otherwise manages an inventory 1152 comprising a plurality of inventory items 1154. The inventory 1152 may be managed on a BSP computing system as a set of electronic records, each record representing an actual inventory item, which is a product or service offered by the BSP, as previously discussed. Each BSP 1150 may uniquely reference inventory items 1154 in its own inventory 1152, such as by using a unique name or other nomenclature. A given BSP's reference or naming of an inventory item 1154 may be slightly different from or varied from the reference or naming of the same item by a BMP in its benefits plans. Accordingly, a mapping of the inventory items 1154 to benefits offered under each benefits plan of each BMP may map inventory maintained by the BSP 1150 with benefits data of the BMPs 1110, 1120, 1130, 1140.

A connector tool 1160 of the PPIS 1100 in FIG. 11 may be an administrative connector that acquires benefits plan data 1111, 1121, 1131, 1141 (for benefits plans of one or more BMPs 1110, 1120, 1130, 1140) and inventory items 1154 (from a BSP) and then correlates the inventory items 1154 to the benefits plan data 1111, 1121, 1131, 1141. The connector tool 1160 may interface in electrical communication with the BSP 1150 and one or more of the BMPs 1110, 1120, 1130, 1140 (e.g., over a communication network). The connector tool 1160 may acquire 1168 or otherwise receive the categorization 1112, 1122, 1132, 1144 and business logic 1114, 1124, 1134, 1144 from the BMPs 1110, 1120, 1130, 1140. The categorization 1112, 1122, 1132, 1142 may provide data related to nomenclature used by the particular BMP 1110, 1120, 1130, 1140, the nomenclature detailing a particular benefit. In the area of vision care, as a non-limiting example, the categorization may provide nomenclature used by the particular BMP to describe or identify each type of eye exam, each type of frame, each specific brand and model of frame, each type of lens, each type of contact lens, each feature which may be added to an exam, frame, lens, etc. The business logic 1114, 1124, 1134, 1144, in a similar way, may comprise details related to determining eligibility of an insured for a particular benefit, and to determining an insurance contribution amount or rate, a discount amount or rate, a copay amount or rate, a frequency of a service or product provision, etc. Continuing the non-limiting example in the vision care arena, business logic may set out that an eye-health and vision correction exam may be performed annually or every twelve months with an insurance contribution of $100, and a co-pay of $35 or 50% of any residual amount above the insurance contribution. Both categorization 1112, 1122, 1132, 1142 and business logic 1114, 1124, 1134, 1144 may be expressed in mathematic terms (equations, etc.), in verbiage, or a combination of these. The connector tool 1160 may acquire 1168 the categorization 1112, 1122, 1132, 1142 and business logic 1114, 1124, 1134, 1144 at the same time or at different times, and may do so periodically.

The connector tool 1160 may electrically communicate with the BSP 1150 to acquire the inventory 1152. The connector tool 1160 may apply various computer-based logic to compare each inventory item 1154 of the inventory 1152 with the nomenclature of the categorization 1112, 1122, 1132, 1142 and relevant business logic 1114, 1124, 1134, 1144 and place each matching inventory item 1154 into a master list 1172, the master list 1172 reflecting the correlation of each inventory item 154 to its appropriate categorization 1112, 1122, 1132, 1142. If the connector tool 1160 cannot, using computer-based logic, match an inventory item 1154 to any nomenclature of the categorization 1112, 1122, 1132, 1142 or business logic 1114, 1124, 1134, 1144, the connector tool 1160 may facilitate human intervention to match the inventory item 1154 appropriately. The master list 1172 is available for use as further described in conjunction with FIG. 12. In one embodiment, the connector tool 1160 may assemble 1176 the business logic 1114, 1124, 1134, 1144 of each of the BMPs 1110, 1120, 1130, 1140 to a logic library 1180. The logic library 1180 then comprises a plurality of logic rules 1183 whereby each benefit of each benefits plan of each BMP is made available for use as further described in conjunction with FIG. 12. In one embodiment, the connector tool 1160 may comprise an inventory connector module 1162 and a logic connector module 1164. The inventory connector module 1162 may be an inventory mapping engine (e.g., an application programming interface (“API” or other software module, firmware module, etc.) to acquire inventory data from a computing device of a BSP, the inventory data including one or more inventory items (e.g., services, products, etc.) provided, offered, or available from the BSP to the individual, including a price (such as a retail price) for each of the one or more inventory items. In one embodiment, the functionality of the inventory connector module 1162 and the logic connector module 1164 may not be discrete modules. In one embodiment, the collection of business logic 1114, 1124, 1134, 1144 may be performed by a logic connector module 1165 external to the connector tool 1160. The external logic connector module 1169 may acquire 1169 the business logic 1114, 1124, 1134, 1144 from each of the BMPs 1110, 1120, 1130, 1140, and may then generate or update 1177 the logic library 1180.

FIG. 12 is a relational diagram of a PPIS 1200, according to another embodiment. The PPIS 1200 of FIG. 12 may be similar in at least some respects to the PPISs of FIGS. 1-11. FIG. 12 illustrates the PPIS 1200 providing an eligibility assessment, according to an embodiment of the present disclosure. A BSP 1203 is shown, having an inventory 1206 (e.g., analogous to the inventory 1152 of FIG. 11). The BSP 1203 may employ a point-of-sale module (POSM) 1209 of the PPIS 1200, such as over a network (e.g., the Internet) via a web page, a mobile app or the like. An individual 1212 (e.g., a patient) may present at the BSP 1203 to obtain a service or product that may be related to a benefit under a benefits plan under which the individual 1212 has coverage. A plurality of BMPs, BMP A 1215, BMP B 1224, BMP C 1236 (e.g., which may be analogous to any of the BMPs 1110, 1120, 1130, 1140 of FIG. 11) are shown in FIG. 12 and the number shown is for convenience of the disclosure and not by way of limitation; more or fewer BMPs may be similarly involved with the PPIS 1200. The BMP A 1215 administers a plurality of benefits plans 1218. Likewise, the BMP B 1524 and the BMP C 1533 each administers a plurality of benefits plans 1227, 1236, respectively.

The PPIS 1200 comprises an eligibility assay 1242, which may further include eligibility matching 1245 and a master list 1248. The master list 1248 may be compiled by acquiring 1251 benefits plan data for benefits plans 1218, 1227, 1236 from the BMPs 1215, 1224, 1233, respectively and acquiring 1254 the inventory 1206 of the BSP 1203, as described in conjunction with FIG. 11. The benefits plan data for the benefits plans 1218, 1227, 1236 may include business logic. The business logic may in some embodiments be derived or otherwise determined from the benefits plan data for the benefits plans 1218, 1227, 1236, as described above. Although not shown in FIG. 12, the benefits plan data for the benefits plans 1218, 1227, 1236 may include categorization for one or more benefits included in the benefits plans 1218, 1227, 1236. (The categorization for the one or more benefits may also be derived or otherwise determined from the benefits plan data for the benefits plans 1218, 1227, 1236, as described above).

The BSP 1203 may obtain or otherwise receive personally identifiable information (PII) 1257 from the individual 1212, and may electrically communicate 1260 the PII 1257 to the eligibility matching 1245. Said otherwise, eligibility matching 1245 may receive, from the BSP, identifying information for an individual. The eligibility matching 1245 may electrically communicate with the BMP A 1215, the BMP B 1224, and the BMP C 1233. Based on the PII 1257, the eligibility matching 1245 may acquire 1263 from each of the BMPs 1215, 1224, 1233 any benefits plan(s) from the pluralities of benefits plans 1218, 1227, 1236 that provide a benefit for the individual 1212 identified by the PII 1257. More particularly, eligibility matching 1245 may acquire 1263 each particular, individualized benefits plan under which the individual 1212 may obtain a benefit. In other words, any particular benefits plan covering the individual 1212 (e.g., a currently valid benefits plan that is in effect) is acquired by eligibility matching 1245. Eligibility matching 1245, employing various computer logic algorithms, extracts from each particular benefits plan (that provides coverage for the individual 1212) nomenclature that identifies benefits and benefit calculations applicable to the individual 1212 under each benefits plan of the pluralities of benefits plans 1218, 1227, 1236. The extracted nomenclature is processed 1266 using the master list 1248. The processing 1266 may comprise identifying each particular benefit under each benefits plan, matching each particular benefit to each relevant inventory item of the inventory 1206 of the BSP 1203 as previously categorized to the master list 1248, deriving any cost-related formulation and/or nomenclature and applying it to provide cost-related calculations. The cost-related calculation may include or otherwise consider, for each benefit (under each applicable benefits plan) and inventory item (from the inventory 1206 of the BSP 1203), an insurance contribution amount or rate, a discount amount or rate, a co-pay amount or rate, and any other formulation or calculation related to providing a cost for each benefit available at the BSP 1203 (or each inventory item in the inventory 1206 of the BSP 1203), including a cost for any inventory item for which no benefit is available under any benefit plan of the pluralities of benefits plans 1218, 1227, 1236 providing coverage for the individual 1212. Eligibility matching 1245 then generates correlated benefits plan data (CBD) 1269. In other words, eligibility matching 1245 may correlate the inventory data (of the BSP) to the benefits plan data to generate structured benefits plan data in which each inventory item of the one or more inventory items is correlated to a benefit of the one or more benefits covered by the one or more benefits plans and a corresponding level of coverage for a corresponding benefits plan. In some embodiments, the structured benefits plan data may be presentable on a display in a uniform manner regardless of the presentation format of the benefits plans from each BMP, thereby affording ease of review by a user of the PPIS 1200. The CBD 1269 may include every benefit of each benefits plan from the pluralities of benefits plans 1218, 1227, 1236 under which the individual 1212 may obtain benefits, calculation data applicable to each inventory item in the inventory 1206 of the BSP, and calculation data determining each cost for each inventory item. The CBD 1269 may further comprise an electronic copy of each benefits plan of the pluralities of benefits plans 1218, 1227, 1236 under which the individual 1212 obtains coverage. The CBD 1269 may be used to generate a “snapshot,” or eligibility history data (EHD) 1275 reflecting every benefit, inventory item, and cost under each benefits plan as of the date when the CBD 1269 is generated. The CBD 1269 may be electrically communicated 1272 to the POSM 1209 for further use of the BSP 1203 in providing one or more inventory items of the inventory 1206 for the individual 1212. In other words, the CBD 1269 provides the foundation for the functions, interfaces, and operations discussed in conjunction with FIGS. 3-10. The EHD 1275 may also be electrically transmitted 1278 to the BSP 1203, as well as being stored by the PPIS 1200. The EHD 1275 may be useful to the BSP 1203 at a future time if a claim is denied by a relevant BMP 1215, 1224, 1233

### EXAMPLES

Some examples of embodiments of the present disclosure are provided here.

### Example 1

A system of determining benefits of an individual, comprising: a network interface to facilitate electronic communication (e.g., over a communication network) with one or more computing systems, including to: receive, at the system, electronic benefits plan data from one or more computing systems of a plurality of benefits management providers (BMPs), the benefits plan data including one or more benefits plans of each of the plurality of BMPs, each benefits plan indicating one or more benefits covered by the benefits plan and indicating a manner of determining a level of contribution (of the plurality of BMPs) for each of the one or more benefits; and receive, at the system, inventory data from one or more benefits service providers (BSPs), the inventory data including one or more inventory items provided by the one or more BSPs to individuals; a memory to store: logic rules (or business logic) that may be unique (or specific) to each BMP that can be used to read from a given benefits plan of the BMP the manner of determining the level of contribution (of the BMP) for each of the one or more benefits included in the given benefits plan and to derive a benefits formula based on the manner of determining the level of contribution; and structured benefits plan data in which each inventory item of the one or more inventory items is mapped to a benefit of the one or more benefits covered by the one or more benefits plans; and one or more processors to: derive, from the benefits plan data, the logic rules (or business logic) and store the logic rules in the memory; correlate the inventory data to the benefits plan data to generate the structured benefits plan data and store the structured benefits plan data in the memory; receive identifying information for an individual; receive an inventory item option for the individual indicating a given inventory item provided by a given BSP; obtain a personal benefits plan of the individual using the identifying information (e.g., a personal benefits plan provided by a BMP of the plurality of BMPs); read the personal benefits plan of the individual (on the fly, at runtime, substantially in real-time) using the logic rules corresponding to the BMP of which the personal benefits plan is from and the structured benefits plan data]] to derive a benefit formula for each benefit included in the personal benefits plan; determine a personalized level of contribution (by or of the BMP) for the given benefit for the individual under the personal benefits plan; and provide, based on a price (e.g., a wholesale or retail price) of the inventory data and the personalized level of contribution (of the BMP), a balance to be paid by the individual for the inventory item option.

### Example 2

The system of Example 1, wherein the one or more processors are further configured to determine, based on the identifying information for the individual, that the individual is eligible for benefit coverage under a benefits plan from the plurality of BMPs.

### Example 3

The system of Example 1, wherein the one or more processors correlating the inventory data to the benefits plan data comprises: generating a master list that correlates each inventory item of the one or more inventory items to a benefit of the one or more benefits.

### Example 4

The system of Example 3, wherein the master list also correlates each benefit of the one or more benefits to one or more of the one or more inventory items.

### Example 5

The system of Example 1, wherein the identifying information for the individual is received from the given BSP.

### Example 6

The system of Example 1, the one or more processors further to: determine a personalized level of contribution (by the BMP) for every inventory item of the given BSP; and provide, based on a price of the inventory data and the personalized level of contribution, a balance (e.g., a personal level of financial responsibility) to be paid by the individual for each inventory item of inventory of the given BSP.

### Example 7

The system of Example 6, the one or more processors further to: determine a personal level of financial responsibility of the individual for every inventory item of the given BSP, based on the retail price and a corresponding personalized level of contribution (by the BMP).

### Example 8

The system of Example 6, the one or more processors further to: determine a personalized level of contribution for every benefit under the personal benefits plan; and provide, based on the retail price of the inventory data and the personalized level of contribution, a balance to be paid by the individual for each and every benefit.

### Example 9

The system of Example 6, wherein the price is a retail price.

### Example 10

The system of Example 1, wherein the balance to be paid by the individual for the inventory item option includes a co-pay amount.

### Example 11

The system of Example 1, wherein the balance to be paid by the individual for the inventory item is personalized pricing for the individual according to the personal benefits plan.

### Example 12

The system of Example 1, wherein the one or more processors provide the balance to be paid by the individual for the inventory item option by providing the balance to be rendered on a graphical user interface.

### Example 13

The system of Example 1, wherein the one or more processors provide the balance to be paid by the individual for the inventory item option by providing the balance to the given BSP.

### Example 14

The system of Example 1, wherein the identifying information for the individual comprises: first name; last name; and date of birth.

### Example 15

The system of Example 14, wherein the identifying information for the individual further comprises at least a portion of a unique identifier (ID) of the individual (e.g., last 4 of SSN or unique member ID).

### Example 16

The system of Example 1, wherein the inventory data is received by: acquiring the inventory data from the electronic health record (EHR) system of each of the one or more BSPs and storing a local copy of the inventory data of each BSP; and syncing the local copy of the inventory data with the EHR system with appropriate frequency to provide near-real-time accuracy of the inventory data of each BSP, wherein the correlating the inventory to the benefits plan data comprises correlating the local copy of the inventory data to the benefits plan data.

### Example 17

The system of Example 1, wherein the one or more processes reading the personal benefits plan comprises: using the logic rules and the structured benefits plan data to correlate personal benefits plan data read from the personal benefits plan with inventory items of the BSP to generate personal structured benefits plan data including the benefit formula for each benefit included in the personal benefit plan, wherein the personalized level of contribution is determined based on the personal structured benefits plan data.

### Example 18

A computer-implemented method of determining an individual's benefits, comprising: acquiring, at a computing system (e.g., over a communications network, electronic benefits plan data from one or more computing systems of a plurality of benefits management providers (BMPs), the benefits plan data including one or more benefits plans of each of the plurality of BMPs, each benefits plan indicating one or more benefits covered by the benefits plan and indicating a manner of determining a level of contribution (of the plurality of BMPs) for each of the one or more benefits; determining (e.g., deriving), by one or more processors, from the benefits plan data, logic rules (e.g., business logic) unique (or specific) to each BMP that can be used to read from a given benefits plan of the BMP the manner of determining the level of contribution for each of the one or more benefits included in the given benefits plan and to derive a benefits formula based on the manner of determining the level of contribution; acquiring, at the computing system (e.g., over the communications network), inventory data from one or more computing systems of one or more benefits service providers (BSPs), the inventory data including one or more inventory items provided by the one or more BSPs to individuals; correlating, by the one or more processors, the inventory data to the benefits plan data to generate structured benefits plan data that maps of each inventory item of the one or more inventory items to a benefit of the one or more benefits covered by the one or more benefits plans and a corresponding level of contribution; receiving identifying information for an individual; receiving at the computing system (e.g., over the communications network), an inventory item option for the individual indicating a given inventory item provided by a given BSP; obtaining, at the computing system (over the communications network), a personal benefits plan of the individual using the identifying information, the personal benefits plan provided by a BMP of the plurality of BMPs; reading (e.g., extracting or otherwise deriving), by the one or more processors, the personal benefits plan of the individual (e.g., on the fly, at run-time, substantially in real-time) using the logic rules corresponding to the BMP of which the personal benefits plan is from and the structured benefits plan data to derive a benefit formula for each benefit included in the personal benefit plan; determining, by the one or more processors, a personalized level of contribution of the BMP for the given benefit for the individual under the personal benefits plan; and providing, based on a price (e.g., a retail price, a wholesale price, a cost) of the inventory data and the personalized level of contribution, a balance to be paid by the individual for the inventory item option.

### Example 19

The method of Example 18, further comprising determining eligibility of the individual under a benefits plan by: determining, by the one or more processors, based on the identifying information for the individual, that the individual is eligible for benefit coverage under a benefits plan from the plurality of BMPs.

### Example 20

The method of Example 19, wherein determining the individual is eligible comprises: electrically communicating with one or more of the plurality of BMPs to obtain the personal benefits plan and confirm that it is in effect.

### Example 21

The method of Example 18, wherein reading the personal benefits plan comprises: using the logic rules and the structured benefits plan data to correlate personal benefits plan data read from the personal benefits plan with inventory items of the BSP to generate personal structured benefits plan data including the benefit formula for each benefit included in the personal benefit plan, wherein the personalized level of contribution is determined based on the personal structured benefits plan data.

### Example 22

The method of Example 18, wherein correlating, by the one or more processors, the inventory data to the benefits plan data further comprises: generating a master list correlating each inventory item of the one or more inventory items to a benefit of the one or more benefits.

### Example 23

The method of Example 22, wherein the master list also correlates each benefit of the one or more benefits to one or more of the one or more inventory items.

### Example 24

The method of Example 18, wherein the identifying information for the individual is received from the given BSP.

### Example 25

The method of Example 24, further comprising: determining, by the one or more processors, a personalized level of contribution (by the BMP) for every item of inventory of the given BSP; and providing, based on a price of the inventory data and the personalized level of contribution, a balance (e.g., a personal level of financial responsibility) to be paid by the individual for every item of inventory of the given BSP.

### Example 26

The method of Example 25, further comprising: determining, by the one or more processors, a personal level of financial responsibility of the individual for every item of inventory of the given BSP, based on the price and a corresponding personalized level of contribution (by the BMP).

### Example 27

The method of Example 25, further comprising: determining a personalized level of contribution for every benefit under the personal benefits plan; and providing, based on the price of the inventory data and the personalized level of contribution, a balance to be paid by the individual for every benefit.

### Example 28

The method of Example 25, wherein the price is a retail price.

### Example 29

The method of Example 18, wherein the balance to be paid by the individual for the inventory item option includes a co-pay amount.

### Example 30

The method of Example 18, wherein determining a personal level of coverage is further based on the identifying information of the individual.

### Example 31

The method of Example 18, wherein providing a balance to be paid by the individual for the inventory item option comprises rendering the balance on a graphical user interface.

### Example 32

The method of Example 18, wherein providing a balance to be paid by the individual for the inventory item option comprises providing to the given BSP the balance to be paid.

### Example 33

The method of Example 18, wherein the acquiring the inventory data comprises: acquiring the inventory data from the electronic health record (EHR) system of each of the one or more BSPs and storing a local copy of the inventory data of each BSP; and syncing the local copy of the inventory data with the EHR system with appropriate frequency to provide near-real-time accuracy of the inventory data of each B SP, wherein the correlating the inventory to the benefits plan data comprises correlating the local copy of the inventory data to the benefits plan data.

### Example 34

The method of Example 18, further comprising: acquiring or otherwise receiving tax data, which is considered when providing the balance to be paid by the individual for the inventory item option.

### Example 35

The method of Example 18, wherein the structured benefits plan data comprises, for each covered benefit: a benefit type (e.g., procedure, service, appliance, etc.); an associated copay; and one or more of limit(s), restriction(s), requirement(s), and frequency.

### Example 36

A system to determine an amount owed for a benefit product or service provided to a given individual, comprising: a network interface to facilitate electronic communication (e.g., over a communication network) with one or more computing systems; a memory to store business logic and a master list; one or more processors operably coupled to the memory; an administrative connector (e.g., a connector tool) to, by the one or more processors: acquire inventory data from a computing device of each of one or more benefits service providers (BSPs) (e.g., Vision Care Providers), the inventory data including one or more inventory items (e.g., services and products (of a BSP)) provided (or offered or available) by the one or more BSPs to individuals, including a price for each of the one or more inventory items; acquire benefits plan data from a computing system of each of a plurality of benefits management providers (BMPs) (e.g., insurers—EyeMed, Davis, Spectera, etc.), the benefits plan data including one or more available benefits plans of each of the plurality of BMPs, each available benefits plan indicating one or more covered benefits (e.g., services and products of a BSP—Exams, Lenses, Frames, Contacts) and a level of coverage for each of the one or more covered benefits; and correlate (e.g., create a mapping) the inventory data to the benefits plan data to generate structured benefits plan data in which each inventory item is correlated to a covered benefit; a co-pay calculator to, by the one or more processors: receive identifying information for an individual; receive an inventory item option for the individual; obtain a personal benefits plan of the individual using the identifying information, the personal benefits plan provided by a BMP of the plurality of BMPs; read the personal benefits plan of the individual (at run-time) using the logic rules corresponding to the BMP of which the personal benefits plan is from and the structured benefits plan data to derive a benefit formula for each benefit included in the personal benefits plan; determine a personal level of coverage of the inventory item option for the individual under the one or more corresponding benefits plans of the one or more available benefits plans of the plurality of BMPs; and provide, based on the price of the inventory data and the personal level of coverage, a balance to be paid by the individual for the inventory item option.

### Example 37

The system of Example 36, wherein the co-pay calculator is further to determine, based on the identifying information for the individual, whether the individual is eligible for coverage, as a benefit, for the inventory item option under one or more corresponding benefits plans of the one or more available benefits plans of the plurality of BMPs.

### Example 38

The system of Example 36, wherein the co-pay calculator is further to: receive the identifying information for the individual from the computing device of a given BSP of the one or more BSPs; and receive the inventory item option for the individual from the computing device of the given BSP.

### Example 39

The system of Example 38, wherein the co-pay calculator is further to provide the balance to be paid by the individual for the inventory item option to the computing device of the given BSP.

### Example 40

The system of Example 36, wherein the co-pay calculator is further to: receive the identifying information for the individual from a user client computing device; and receive the inventory item option for the individual from the user client computing device.

### Example 41

The system of Example 40, wherein the co-pay calculator is further to provide the balance to be paid by the individual for the inventory item option to the user client computing device.

### Example 42

A computer-implemented method of rendering a listing of benefits according to a personal benefits plan of a given individual, comprising: acquiring benefits plan data from a plurality of benefits management providers (BMPs) (e.g., insurers—EyeMed, Davis, Spectera, etc.), the benefits plan data including one or more available benefits plans of each of the plurality of BMPs, each available benefits plan indicating one or more covered benefits (e.g., services and products of a BSP—Exams, Lenses, Frames, Contacts) and a manner of determining (e.g., a formulary for determining) a level of contribution for each of the one or more covered benefits; determining (e.g., extracting, deriving, by one or more processors, from the benefits plan data, logic rules (or business logic) that may be unique and/or specific to each BMP of the plurality of BMPs, the logic rules to be used to read from a given benefits plan of the BMP the manner of determining the level of contribution (of the BMP) for each of the one or more benefits included in the given benefits plan and to derive a benefit formula based on the manner of determining the level of contribution; acquiring inventory data from one or more benefits service providers (BSPs) (e.g., Vision Care Providers), the inventory data including one or more inventory items (e.g., services and products (of a BSP)) provided (or otherwise offered or made available) by the one or more BSPs to individuals, including a price for each of the one or more inventory items; correlating (e.g., creating a mapping) the inventory data to the benefits plan data to generate structured benefits plan data in which each inventory item is correlated to a covered benefit; receiving identifying information for an individual; receiving a plurality of inventory item options for the individual; obtaining a personal benefits plan of the individual using the identifying information, the personal benefits plan provided by a BMP of the plurality of BMPs; reading (e.g., extracting or deriving at runtime), by the one or more processors, the personal benefits plan of the individual (on the fly, at run-time, substantially in real-time) using the logic rules (corresponding to the BMP of which the personal benefits plan is from and the structured benefits plan data to derive a benefit formula for each benefit included in the personal benefit plan; determining, based on the identifying information for the individual, the individual is eligible for each of the plurality of inventory item options under one or more corresponding benefits plans of the one or more available benefits plans of the plurality of BMPs; determining for each of the plurality of inventory item options a personalized level of contribution (of the BMP) for each inventory item option for the individual under the one or more corresponding benefits plans; determining, based on the price of the inventory data and the personal level of coverage, a balance to be paid by the individual for each of the plurality of inventory item options; and rendering a listing of the plurality of options, the listing including indication of each option of the plurality of options and a corresponding balance to be paid by the individual for each option.

The phrases “connected to” and “coupled to” are used herein in their ordinary sense, and are broad enough to refer to any suitable coupling or other form of interaction between two or more entities, including mechanical, fluid, and thermal interaction. Two components may be coupled to each other even though they are not in direct contact with each other. The phrase “attached to” refers to interaction between two or more entities which are in direct contact with each other and/or are separated from each other only by a fastener of any suitable variety (e.g., an adhesive, etc.).

The term “opposite” is a relational term used herein to refer to a placement of a particular feature or component in a position corresponding to another related feature or component wherein the corresponding features or components are positionally juxtaposed to each other. By way of example, a person's right hand is opposite the person's left hand.

The terms “a” and “an” can be described as one, but not limited to one. For example, although the disclosure may recite an element having, e.g., “a line of stitches,” the disclosure also contemplates that the element can have two or more lines of stitches.

Unless otherwise stated, all ranges include both endpoints and all numbers between the endpoints.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment. Not every embodiment is shown in the accompanying illustrations, however, at least a preferred embodiment is shown. At least some of the features described for a shown preferred embodiment are present in other embodiments.

Furthermore, the described features, operations, or characteristics may be arranged and designed in a wide variety of different configurations and/or combined in any suitable manner in one or more embodiments. Thus, the detailed description of the embodiments of the systems and methods is not intended to limit the scope of the disclosure, as claimed, but is merely representative of possible embodiments of the disclosure. In addition, it will also be readily understood that the order of the steps or actions of the methods described in connection with the embodiments disclosed may be changed as would be apparent to those skilled in the art. Thus, any order in the drawings or Detailed Descriptions is for illustrative purposes only and is not meant to imply a required order, unless specified to require an order.

Embodiments may include various steps, which may be embodied in machine-executable instructions to be executed by a general-purpose or special-purpose computer (or other electronic device). Alternatively, the steps may be performed by hardware components that include specific logic for performing the steps, or by a combination of hardware, software, and/or firmware.

Embodiments may also be provided as a computer program product including a computer-readable storage medium having stored instructions thereon that may be used to program a computer (or other electronic device) to perform processes described herein. The computer-readable storage medium comprises at a non-transient storage medium, and may include, but is not limited to a hard drive, a fixed disk, a removable disk, an optical disk, a CD-ROM, a CD-RW, a DVD-ROM, a DVD-RW, a read-only memory (ROM), a random access memory (RAM), an erasable programmable ROM (EPROM), an electrically erasable programmable ROM (EEPROM), a magnetic card, an optical card, a solid-state memory device, or other types of media/machine-readable media suitable for storing electronic instructions.

A software module, module, or component may include any type of computer instruction or computer executable code located within a memory device and/or computer-readable storage medium, as is well-known in the art.

It will be obvious to those having skill in the art that many changes may be made to the details of the above described embodiments without departing from the underlying principles of the invention. The scope of the present invention should, therefore, be determined only by the following claims.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having reasonable skill in the art that changes may be made to the details of the above-described embodiments without departing from the underlying principles of the invention. Embodiments of the invention in which an exclusive property or privilege is claimed are defined as follows.

1. A system of determining benefits of an individual, comprising:
a network interface to facilitate electronic communication with one or more computing systems, including to:
receive benefits plan data from a plurality of benefits management providers (BMPs), the benefits plan data including one or more benefits plans of each of the plurality of BMPs, each benefits plan indicating one or more benefits covered by the benefits plan and indicating a manner of determining a level of contribution for each of the one or more benefits; and
receive inventory data from one or more benefits service providers (BSPs), the inventory data including one or more inventory items provided by the one or more BSPs to individuals;
a memory to store:
logic rules for each BMP that can be used to read from a given benefits plan of the BMP the manner of determining the level of contribution for each of the one or more benefits included in the given benefits plan and to derive a benefits formula based on the manner of determining the level of contribution; and
structured benefits plan data in which each inventory item of the one or more inventory items is mapped to a benefit of the one or more benefits covered by the one or more benefits plans; and
one or more processors to:
derive, from the benefits plan data, the logic rules and store the logic rules in the memory;
correlate the inventory data to the benefits plan data to generate the structured benefits plan data;
receive identifying information for an individual;
receive an inventory item option for the individual indicating a given inventory item provided by a given BSP;
obtain a personal benefits plan of the individual using the identifying information;
read the personal benefits plan of the individual using the logic rules to derive a benefit formula for each benefit included in the personal benefits plan;
determine a personalized level of contribution for the given benefit under the personal benefits plan; and
provide, based on the personalized level of contribution, a balance to be paid by the individual for the inventory item option.

2. The system of claim 1, wherein the one or more processors are further configured to determine, based on the identifying information for the individual, that the individual is eligible for benefit coverage under a benefits plan from the plurality of BMPs.

3. The system of claim 1, wherein the one or more processors correlating the inventory data to the benefits plan data comprises:
generating a master list that correlates each inventory item of the one or more inventory items to a benefit of the one or more benefits.

4. The system of claim 1, wherein the identifying information for the individual is received from the given BSP.

5. The system of claim 1, the one or more processors further to:
determine a personalized level of contribution for every inventory item of the given BSP; and
provide, based on a price of the inventory data and the personalized level of contribution, a balance to be paid by the individual for each inventory item of inventory of the given BSP.

6. The system of claim 1, wherein the balance to be paid by the individual for the inventory item option includes a co-pay amount.

7. The system of claim 1, wherein the one or more processors provide the balance to be paid by the individual for the inventory item option by providing the balance to be rendered on a graphical user interface.

8. The system of claim 1, wherein the one or more processors provide the balance to be paid by the individual for the inventory item option by providing the balance to the given BSP.

9. The system of claim 1, wherein the inventory data is received by:
acquiring the inventory data from the electronic health record (EHR) system of each of the one or more BSPs and storing a local copy of the inventory data of each BSP; and
syncing the local copy of the inventory data with the EHR system with appropriate frequency to provide near-real-time accuracy of the inventory data of each BSP,
wherein the correlating the inventory to the benefits plan data comprises correlating the local copy of the inventory data to the benefits plan data.

10. The system of claim 1, wherein the one or more processes reading the personal benefits plan comprises:
using the logic rules and the structured benefits plan data to correlate personal benefits plan data read from the personal benefits plan with inventory items of the BSP to generate personal structured benefits plan data including the benefit formula for each benefit included in the personal benefit plan,
wherein the personalized level of contribution is determined based on the personal structured benefits plan data.

11. A computer-implemented method of determining an individual's benefits, comprising:
acquiring, at a computing system, electronic benefits plan data from a plurality of benefits management providers (BMPs), the benefits plan data including one or more benefits plans of each of the plurality of BMPs, each benefits plan indicating one or more benefits covered by the benefits plan and indicating a manner of determining a level of contribution for each of the one or more benefits;
determining, by one or more processors, from the benefits plan data, logic rules that can be used to read from a given benefits plan of the BMP the manner of determining the level of contribution for each of the one or more benefits included in the given benefits plan and to derive a benefits formula based on the manner of determining the level of contribution;
acquiring, at the computing system, inventory data from one or more benefits service providers (BSPs), the inventory data including one or more inventory items provided by the one or more BSPs to individuals;
correlating, by the one or more processors, the inventory data to the benefits plan data to generate structured benefits plan data that maps of each inventory item of the one or more inventory items to a benefit of the one or more benefits covered by the one or more benefits plans;
receiving identifying information for an individual;
receiving at the computing system, an inventory item option for the individual indicating a given inventory item provided by a given BSP;
obtaining, at the computing system, a personal benefits plan of the individual using the identifying information;
reading, by the one or more processors, the personal benefits plan of the individual using the logic rules to derive a benefit formula for each benefit included in the personal benefit plan;
determining, by the one or more processors, a personalized level of contribution for the given benefit under the personal benefits plan ; and
providing, based on the personalized level of contribution, a balance to be paid by the individual for the inventory item option.

12. The method of claim 11, further comprising determining eligibility of the individual under a benefits plan by:
determining, by the one or more processors, based on the identifying information for the individual, that the individual is eligible for benefit coverage under a benefits plan from the plurality of BMPs.

13. The method of claim 11, wherein reading the personal benefits plan comprises:
using the logic rules and the structured benefits plan data to correlate personal benefits plan data read from the personal benefits plan with inventory items of the BSP to generate personal structured benefits plan data including the benefit formula for each benefit included in the personal benefit plan,
wherein the personalized level of contribution is determined based on the personal structured benefits plan data.

14. The method of claim 11, wherein correlating, by the one or more processors, the inventory data to the benefits plan data further comprises:
generating a master list correlating each inventory item of the one or more inventory items to a benefit of the one or more benefits.

15. The method of claim 11, wherein the identifying information for the individual is received from the given BSP.

16. The method of claim 15, further comprising:
determining, by the one or more processors, a personalized level of contribution for every item of inventory of the given BSP; and
providing, based on a price of the inventory data and the personalized level of contribution, a balance to be paid by the individual for every item of inventory of the given BSP.

17. The method of claim 11, wherein the balance to be paid by the individual for the inventory item option includes a co-pay amount.

18. The method of claim 11, wherein providing a balance to be paid by the individual for the inventory item option comprises rendering the balance on a graphical user interface.

19. The method of claim 11, wherein providing a balance to be paid by the individual for the inventory item option comprises providing to the given BSP the balance to be paid.

20. The method of claim 11, wherein the acquiring the inventory data comprises:
acquiring the inventory data from the electronic health record (EHR) system of each of the one or more BSPs and storing a local copy of the inventory data of each BSP; and
syncing the local copy of the inventory data with the EHR system with appropriate frequency to provide near-real-time accuracy of the inventory data of each BSP,
wherein the correlating the inventory to the benefits plan data comprises correlating the local copy of the inventory data to the benefits plan data.
